(19) United States
(12) Patent Application Publication
(10) Pub. No.: US 2009/0264457 A1
(43) Pub. Date: Oct. 22, 2009

(54) Combination of at least two 5HT6-Ligands

(76) Inventors: Xavier Codony-Soler, Mataro (ES); Jose-Miguel Vela-Hernandez, Barcelona (ES); Helmut H. Buschmann, Sant Just Desvern (ES)

Correspondence Address: Cooper & Dunham LLP, 30 Rockefeller Plaza, 20th Floor, New York, NY, 10112, US

(21) Appl. No.: 12/221,364

(22) Filed: Aug. 1, 2008

(30) Foreign Application Priority Data
Aug. 1, 2007 (EP) 07384032.4

(51) Int. Cl. A61K 31/437; A61K 31/404; A61K 31/426; A61K 31/416; A61K 31/454; A61P 25/00; A61P 25/28

(52) U.S. Cl. 514/292; 514/412; 514/368; 514/406; 514/322

(57) ABSTRACT

The present invention relates to a Combination of at least two 5HT6-Ligands of which one is a partial or agonist while the other is a full antagonist or an inverse agonist, a medicament comprising this comination, the use of the combiantion in the manufacture of a medicament for the treatment of memory disorders ADHD, and methods of treatment using the combination or its members.

The present invention relates to a Combination of at least two 5HT6-Ligands of which one is a partial or full agonist while the other is a full antagonist or an inverse agonist, a medicament comprising this comination, the use of the combiantion in the manufacture of a medicament for the treatment of cognintive or degenerative brain disorders, memory disorders, or ADHD, or for memory enhancement, and methods of treatment using the combination or its respective members in a dosing pattern.

Cognitive and/or degenerative brain disorders are characterized clinically by progressive loss of memory, cognition, reasoning, judgement and emotional stability that gradually leads to profound mental deterioration and ultimately death. In an example of such disorders, Alzheimer's disease is a common cause of progressive mental failure (dementia) in aged humans and is believed to represent the fourth most common medical cause of death in the United States. In particular, Alzheimer's disease is associated with degeneration of cholinergic neurons in the basal forebrain that play a fundamental role in cognitive functions, including memory. Cognitive and/or degenerative brain disorders have been observed in varied races and ethnic groups world-wide and presents a major public health problem. These diseases are currently estimated to affect about two to three million individuals in the United States alone and the occurrence will increase world-wide as the human life span increases. On the other hand shortcomings or failure to use the memory to its full abilities is a common problem and thus also often needs pharmaceutical attention In particular, it was an object of the present invention to provide a medicament suitable for the prophylaxis and/or treatment of cognitive disorders or for memory enhancement.

Said object has been achieved by providing a combination of active substances comprising
at least one compound (A),
being selected from compounds binding to the 5HT6-receptor and acting as an full agonist or partial agonist;
and at least one compound (B)
   being selected from compounds binding to the 5HT6-receptor and acting as an antagonist or inverse agonist;

It has now been surprisingly demonstrated that an active substance combination comprising a 5HT6 ligand being a full or partial agonist and a 5HT6 ligand being a full antagonist or inverse agonist (as well as the simultaneous or nearly simultaneous use of these compounds) was able to positively act on the CNS activities in a mammal by acting in models of cognitive disorders especially by enhancing the memory of a mammal.

The superfamily of serotonin receptors (5-HT) includes 7 classes (5-HT₁-5-HT₇) encompassing 14 human subclasses [D. Hoyer, et al., Neuropharmacology, 1997, 36, 419]. The 5-HT₆ receptor is a serotonin receptor identified by molecular cloning both in rats [F. J. Monsma, et al., Mol. Pharmacol., 1993, 43, 320; M. Ruat, et al., Biochem. Biophys. Res. Commun., 1993, 193, 268] and in humans [R. Kohen, et al., J. Neurochem., 1996, 66, 47]. Compounds with 5-HT₆ receptor affinity are useful for the treatment of various disorders of the Central Nervous System and of the gastrointestinal tract, such as irritable intestine syndrome. Compounds with 5-HT₆ receptor affinity are also useful in the treatment of anxiety, depression and cognitive memory disorders [M. Yoshioka, et al., Ann. NY Acad. Sci., 1998, 861, 244; A. Bourson, et al., Br. J. Pharmacol., 1998, 125, 1562; D. C. Rogers, et al., Br. J. Pharmacol. Suppl., 1999, 127, 22P; A. Bourson, et al., J. Pharmacol. Exp. Ther., 1995, 274, 173; A. J. Sleight, et al., Behav. Brain Res., 1996, 73, 245; T. A. Branchek, et al., nnu. Rev. Pharmacol. Toxicol., 2000, 40, 319; C. Routledge, et al., Br. J. Pharmacol., 2000, 130, 1606]. It has been shown that typical and atypical antipsychotic drugs for treating schizophrenia have a high affinity for 5-HT₆ receptors [B. L. Roth, et al., J. Pharmacol. Exp. Ther., 1994, 268, 1403; C. E. Glaff, et al., Mol. Med., 1995, 1, 398; F. J. Mosma, et al., Mol. Pharmacol., 1993, 43, 320; T. Shinkai, et al., Am. J. Med. Genet., 1999, 88, 120]. Compounds with 5-HT₆ receptor affinity are useful for treating infant hyperkinesia (ADHD, attention deficit/hyperactivity disorder) [W. D. Hirst, et al., Br. J. Pharmacol., 2000, 130, 1597; C. Gerard, et al., Brain Research, 1997, 746, 207; M. R. Pranzatelli, Drugs of Today, 1997, 33, 379]. Moreover, it has been shown that the 5-HT₆ receptor also plays a role in food ingestion [Neuropharmacology, 41, 2001, 210-219].

“Compound/s binding to the 5HT6-receptor” (with “5HT6-Ligand” being also used in this description and being one and the same as “Compound/s binding to the 5HT6-receptor”) as used in this application is/are defined as having on the 5HT6-receptor an Kᵢ value of ≦5000 nM. More preferably the “compound/s binding to the 5HT6-receptor” are having on the 5HT6-receptor an Kᵢ value of s; 1000 nM, more preferably ≦500 nM. More preferably, the Kᵢ value is ≦250 nM. More preferably, the Kᵢ value is ≦100 nM. More preferably, the Kᵢ value is ≦100 nM. Most preferably, the Kᵢ value is ≦50 nM. An also fitting definition is to define the compounds by way of their IC₅₀ values and thus “compound/s binding to the 5HT6-receptor” as used in this application is also understood as meaning compounds having on the 5HT6-receptor an IC₅₀ value of ≦5000 nM. More preferably the “compound/s binding to the 5HT6-receptor” are having on the 5HT6-receptor an IC₅₀ value of ≦1000 nM, more preferably of ≦500 nM. More preferably, the IC₅₀ value is ≦250 nM. More preferably, the IC₅₀ value is ≦100 nM. Most preferably, the IC₅₀ value is ≦50 nM. Compound binding to the 5HT6-receptor may be partial agonists, antagonists, full agonists, or inverse agonists. Pharmacological test systems to determine all of these functionalities are well-known in the art.

The active substance combination according to this invention comprises preferably 1-99% by weight of the component (A) and 99-1% by weight of the component (B), more preferably 10-80% by weight of the component (A) and 80-20% by weight of the component (B), these percentages referring to the total weight of both components (A) and (B).

Assays that may be used for determining the affinity and selectivity of a 5-HT7 receptor agonist and/or other affinities to 5-HT receptors are well known in the art and especially measuring the affinities to these receptors are offered by service companies like. It is possible to classify a compound with 5-HT receptor affinity as full or partial agonist (also as inverse agonist or antagonist) according to the reference of S. M. Stahl, Essential Psychopharmacology, Neuroscientific basis and practical applications, Ed. Cambridge, 1996, Chapter 3. The respective part of the literature is hereby incorporated by reference and forms part of the disclosure.

As defined herein it is preferred if the functionality of the compounds (A) or (B) both binding to the 5HT6 receptor, the 5HT6 ligand, is determined in regards to the same biological group of mammals. Thus, to select and define a Compound (A) as being a 5HT6 ligand and acting as a partial or full agonist, and to select and define a Compound (B) as being a 5HT6 ligand and acting as an antagonist or inverse agonist, the same test systems, especially in regards to the species being used, should be employed. So, to determine and define the affinity and functionality of at least one Compound (A) and at least one Compound (B) to be used in the same combination of active substances according to the invention the animals or organs (if non-human), cells, cell systems, nucleic acids, receptors, proteins or peptides used to determine affinity and functionality should be from the same species, e.g. rat, mouse, human.

Especially preferred embodiments of the invention encompass the use of a compound with a very specific binding to the 5HT6 receptor being in its binding profile more specific in its affinity (thus showing a lower Ki) to the 5HT6 receptor than in its affinity to other 5HT Receptors.

Therefore in a preferred embodiment of the invention the compounds (A) and/or (B), preferably both are binding to the SHT6 receptor with a higher affinity—expressed as a lower Ki-value—to the 5HT6 receptor than to the 5-HT1A or 5HT7 receptor; especially binding with an affinity higher by a factor of at least 10, preferably with an affinity higher by a factor of at least 30, more preferably with an affinity higher by a factor of at least 50, most preferably with an affinity higher by a factor of at least 100.

By way of exemplifying the above paragraph a compound X—specific to the 5HT6 receptor-, is assumed to have an affinity—expressed as a Ki value—of 1 nM and an affinity to the 5HT1A receptor of a Ki value of 42 nM and thus would have an affinity to the 5HT6 receptor higher (over the 5HT1A receptor) by a factor of 42.

In another preferred embodiment of the invention the compounds (A) and/or (B), preferably both are binding to the 5HT6 receptor with a Kᵢ value of ≦5000 nM, preferably of ≦1000 nM, more preferably of ≦500 nM. Preferably, the Kᵢ value is ≦250 nM, or ≦100 nM, or most preferably is ≦50 nM.

In another preferred embodiment of the invention the compounds (A) and/or (B), preferably both are binding to the 5HT6 receptor with a higher affinity to the 5HT6 receptor than to the 5-HT1A or 5HT7 receptor; especially binding with an affinity higher by a factor of at least 10, preferably with an affinity higher by a factor of at least 30, more preferably with an affinity higher by a factor of at least 50, most preferably with an affinity higher by a factor of at least 100;

and
the compounds (A) and/or (B), preferably both are binding to the 5HT6 receptor with a K, value of ≦5000 nM, preferably of ≦1000 nM, more preferably of ≦500 nM. Preferably, the Kᵢ value is ≦250 nM, or ≦100 nM, or most preferably is ≦50 nM.

An “agonist” is defined as a compound that binds to a receptor and has an intrinsic effect, and thus, increases the basal activity of a receptor when it contacts the receptor. Full agonists show the maximum effect on the 5-HT6 receptor, whereas a partial agonist is giving less (e.g. 80%) of the response of the full agonist as a maximum.

An “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

An “inverse agonist” is defined as a compound that produces an effect opposite to that of the agonist by occupying the same receptor and, thus, decreases the basal activity of a receptor (i.e., signalling mediated by the receptor). Such compounds are also known as negative antagonists. An inverse agonist is a ligand for a receptor that causes the receptor to adopt an inactive state relative to a basal state occurring in the absence of any ligand. Thus, while an antagonist (or neutral antagonist) can inhibit the activity of an agonist, an inverse agonist is a ligand that can alter the conformation of the receptor in the absence of an agonist.

In another preferred embodiment of the combination of active substances according to the invention at least one compound (A) or at least one compound (B) or at least one compound (A) and one compound (B) binding to the 5HT6-receptor is/are selected from SB-271046, SB-271946 A, Ro 04-6790, SB-357134, SB-399885, SB-399885T, 5-methoxy-2-phenyl-N,N-dimethyl triptamine (BGC20-761), or 2-ethyl-5-methoxy-N,N-dimethyltriptamine, WAY-181187, WAY-466 preferably at least one compound (A) is selected from 2-ethyl-5-methoxy-N,N-dimethyltriptamine, WAY-181187, WAY-466 and/or at least one compound (B) is selected from SB-271046, SB-271046-A Ro 04-6790, SB-357134, SB-399885, SB-399885T, 5-methoxy-2-phenyl-N,N-dimethyl triptamine (BGC20-761).

SB-271046 (5-Chloro-N-(4-methoxy-3-(piperazin-1-yl)phenyl)-3-methyl-2-benzothiophene sulfonamide) is described in Bromidge et al., J. Med. Chem. 48, 353-356 (1999), included here in its entirety by reference.
Ro 04-6790 (4-Amino-N-(2,6 bis-methylamino-pyrimidin-4-yl)benzene sulphonamide is described in Sleight et al., Br. J. Pharmacol. 124, 556-562 (1998), included here in its entirety by reference.
5-methoxy-2-phenyl-N,N-dimethyl triptamine (BGC20-761) is described in detail in Mitchell et al., Neuropharmacol. 50, 412-420 (2006), included here in its entirety by reference.
SB-357134, SB-399885, SB-399885T and SB-271046-A are described and mentioned in Meneses, BehavBrain Res. 118, 107-110, (1991), Perez-Garcia and Meneses, Pharmacol. Biochem. Behav., 81, 673-682 (2005) and Stean et al., Pharmacol. Biochem. Behav., 71, 645-654 (2002) included here in their entirety by reference.
WAY-181187 and WAY-466 are described and mentioned in Beyer at al., Neuropsychopharmacol., 15, S382-S382 (2005) and Schechter et. al., Neuropsychopharmacol., 29, S237-S237 (2004) respectively included here in their entirety by reference

Further selective 5HT6 receptor ligands acting as antagonists can be found at Sleight et al., Br. J. Pharmacol. 124, 556-562 (1998), included here in its entirety by reference.

Further selective 5HT6 receptor ligands acting as agonists (and in some cases also antagonists) can be found in the following articles: Cole et al., J. Med. Chem. 48, 353-356 (2005), Glennon et al., J. Med. Chem., 43, 1011-1018 (2000); Holenz et al., J. Med. Chem., 48, 1781-1795 (2005), Mattson et al., Bioorg. Med. Chem. Lett., 15, 4230-4234 (2005), Schechter et. al., Neuropsychopharmacol., 29, S237-S237 (2004). All of them are included here in their entirety by reference.

In a preferred embodiment of the combination of active substances according to the invention, at least one compound (A) or at least one compound (B) or at least one compound (A) and one compound (B) binding to the 5HT6-receptor is/are selected from sulfonamide compounds according to formula (I)

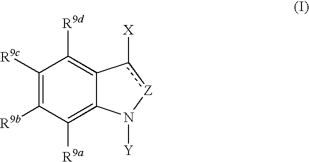

wherein
=Z either represents ═C(R¹⁰) or —CH₂ or ═N;
and wherein
   either
Y represents —S(O₂)—R³, while X represents R¹;
   or
X represents R¹, while Y represents (CH₂)ₙ—R¹¹ or
Y represents R¹, while X represents (CH₂)ₙ—R¹²;
   while
   one of R^{9a}, R^{9b}, R^{9c}, or R^{9d} represents N(R³)—S(O₂)-A, or N—(S(O₂)-A)₂;
and wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—C₁₋₅-alkyl, C(═O)—O—C₁₋₅-alkyl, —O—C(═O)—C₁₋₅-alkyl, oxo (═O), F, Cl, Br, I, —CN, —OCF₃, —SCF₃, —OH, —SH, —NH₂, —NH(C₁₋₅-alkyl), —N(CF₁₋₅-alkyl)₂, —NH—C(═O)—C₁₋₆-alkyl, —N(C₁₋₅-alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy, benzyl, and pyridinyl and which may be condensed with a saturated or unsaturated mono- or bicyclic ring system, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅alkyl, —S(═O)₂-phenyl, cyclopropyl, cyclobutyl, cyclopentyl, cyclohexyl, phenyl, phenoxy and benzyl and whereby the rings of the ring system are 5-6 or 7-membered and may contain 1, 2 or 3 heteroatom(s) independently selected from the group consisting of nitrogen, oxygen and sulfur and which may be bonded via a linear or branched C₁₋₄ alkylene, C₂₋₆ alkenylene or C₂₋₆ alkinylene group which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃ and wherein the heteroaryl radical contains 1, 2 or 3 heteroatom(s) independently selected from the group consisting of nitrogen, oxygen and sulfur as ring member(s);
R¹ represents hydrogen, a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C2 Hr, —NO₂, —CN, —NH—CH₃ and —S—CH₃; or an optionally at least monosubstituted alkyl-aryl radical;
R³ represents a hydrogen atom; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d} —if not N(R³)—S(O₂)-A or N—(S(O₂)-A)₂— independently from one another, each represent a hydrogen atom; —NO₂; —NH₂; —SH; —OH; —CN; —C(═O)—H; —C(═O)—R′; —C(═O)—O—R′; —OR; —SR; —N(R)—S(═O)₂—R′; —NH—R′; —NR′R″; F; Cl, Br; I; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or a 5- to 14-membered aryl or heteroaryl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may be bonded via a linear or branched C₁₋₆ alkylene group and wherein the heteroaryl radical contains 1, 2 or 3 heteroatom(s) independently selected from the group consisting of nitrogen, oxygen and sulfur as ring member(s);
R¹⁰ represents a hydrogen atom; —NO₂; —NH₂; —SH; —OH; —CN; —C(═O)—OH; —O—R′; —S—R′; —C(═O)—OR″; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —S—C₁₋₅-alkyl, oxo (═O)), thioxo (═S), —C(═O)—OH, —C(═O)—O—C₁₋₄-alkyl, —O—C(═O)—C₁₋₆alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and whereby the rings of the ring system are 5-, 6- or 7-membered and may contain 1, 2 or 3 heteroatom(s) as ring member(s) independently selected from the group consisting of nitrogen, oxygen and sulfur;
R¹² represents R¹¹,
or
represents

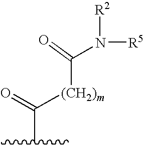

with n being 0;
or represents —C(OC₁₋₄₅-alkyl)-(CH₂)ₘ—R¹¹;
R¹³ represents a saturated or unsaturated, optionally at least mono-substituted cycloaliphatic radical, or —CHR′R″;
n being 0, 1, 2, 3 or 4;
m being 0, 1, 2, 3 or 4;
R′ and R″ identical or different, each represents a saturated or unsaturated, linear or branched, optionally at least mono-substituted aliphatic radical;
R* and R** identical or different, each represents a saturated or unsaturated, linear or branched, optionally at least mono-substituted aliphatic radical; or
R* and R** together with the connecting nitrogen form an optionally at least monosubstituted heterocyclyl radical
R² represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
   or
R² and R⁵ together with the bridging nitrogen form a saturated, unsaturated or aromatic 3- to 9-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—C₁₋₅-alkyl; —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and whereby the rings of the ring system are 5-6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

Aliphatic radicals/groups, as referred to in the present invention, are optionally mono- or polysubstituted and may be branched or unbranched, saturated or unsaturated. Unsaturated aliphatic groups, as defined in the present invention, include alkenyl and alkinyl radicals. Saturated aliphatic groups, as defined in the present invention, include alkyl radicals. Preferred aliphatic radicals according to the present invention include but are not restricted to methyl, ethyl, vinyl (ethenyl), ethinyl, propyl, n-propyl, isopropyl, allyl (2-propenyl), 1-propinyl, methylethyl, butyl, n-butyl, iso-butyl, sec-butyl, tert-butyl butenyl, butinyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl, 1-methylpentyl, n-heptyl, n-octyl, n-nonyl and n-decyl.

In the context of this invention, alkyl radical or group is understood as meaning saturated, linear or branched hydrocarbons, which can be unsubstituted or mono- or polysubstituted. Alkenyl and alkinyl groups, on the other hand include groups like e.g. —CH═CH—CH₃ or —C≡C—CH₃, while the saturated alkyl encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C₁₋₁₀-alkyl and C₁₋₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc.

In the context of this invention cycloalkyl radical or group is understood as meaning saturated and unsaturated (but not aromatic) cyclic hydrocarbons (without a heteroatom in the ring), which can be unsubstituted or mono- or polysubstituted. Furthermore, C-cycloalkyl represents C3- or C4-cycloalkyl, C3-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C4-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. However, mono- or polyunsaturated, preferably monounsaturated, cycloalkyls also in particular fall under the term cycloalkyl as long as the cycloalkyl is not an aromatic system. The alkyl and cycloalkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, and also adamantly.

In the context of this invention alkyl-cycloalkyl is understood as meaning a cycloalkyl group (see above) being connected to another atom through a C₁₋₆alkyl group (see above), whereas the C₁₋₆-alkyl-group is always saturated and unsubstituted, and linear or branched.

In connection with alkyl or aliphatic group—unless defined otherwise—the term substituted in the context of this invention is understood as meaning replacement of at least one hydrogen radical by F, Cl, Br, I, NH₂, SH or OH, “polysubstituted” (more than once substituted) radicals being understood as meaning that the replacement takes effect both on different and on the same atoms several times with the same or different substituents, for example three times on the same C atom, as in the case of CF₃, or at different places, as in the case of e.g. —CH(OH)—CH═CH—CHCl₂. “Optionally at least monosubstituted” means either “monosubstituted”, “polysubstituted” or—if the option is not fulfilled—“unsubstituted”.

The term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂—, etc.

The term “ring system” including “mono- or bicyclic ring system” according to the present invention refers to a ring sytem or ring sytems which comprises or comprise saturated, unsaturated or aromatic carbocyclic ring sytems which contain optionally at least one heteroatom as ring member and which are optionally at least mono-substituted. Said ring systems may be condensed to other carbocyclic ring systems such as aryl groups, naphtyl groups, heteroaryl groups, cycloalkyl groups, etc. Preferably the “ring system” may consist of 1 ring (monocyclic), or 2 (bicyclic) or 3 (tricyclic) rings being condensed.

An aryl radical or group is understood as meaning ring systems with at least one aromatic ring but without heteroatoms even in only one of the rings. Examples are phenyl, naphthyl, fluoranthenyl, fluorenyl, tetralinyl or indanyl, in particular 9H-fluorenyl or anthracenyl radicals, which can be unsubstituted or monosubstituted or polysubstituted.

In the context of this invention alkyl-aryl is understood as meaning an aryl group (see above) being connected to another atom through a C₁₋₆-alkyl-group (see above), whereas the C₁₋₆-alkyl-group is always saturated and unsubstituted, and linear or branched.

A heterocyclyl radical or group is understood as meaning heterocyclic ring systems, saturated or unsaturated ring which contains one or more heteroatoms from the group consisting of nitrogen, oxygen and/or sulfur in the ring and can also be mono- or polysubstituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline.

In the context of this invention alkyl-heterocylyl is understood as meaning a heterocyclyl group (see above) being connected to another atom through a C₁₋₆-alkyl group (see above), whereas the C₁₋₆-alkyl-group is always saturated and unsubstituted, and linear or branched.

In connection with aryl or alkyl-aryl, cycloalkyl or alkyl-cycloalkyl, heterocyclyl or alkyl-heterocyclyl, substituted is understood—unless defined otherwise—as meaning substitution of the ring-system of the aryl or alkyl-aryl, cycloalkyl or alkyl-cycloalkyl; heterocyclyl or alkyl-heterocyclyl by OH, SH, ═O, halogen (F, Cl, Br, I), CN, NO₂, COOH; NRₓR_{y}, with Rₓ, and R_{y} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆alkyl-group; a saturated or unsaturated, linear or branched, substituted or unsubstituted —C(O)—O—C₁₋₆ alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl. “Optionally at least monosubstituted” means either “monosubstituted”, “polysubstituted” or—if the option is not fulfilled—“unsubstituted”.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH4, but in particular (mono)- or (di)sodium, (mono)- or (di)potassium, magnesium or calcium salts.

These physiologically acceptable salts can also be formed with anions or acids and in the context of this invention is understood as meaning salts of at least one of the compounds used according to the invention—usually protonated, for example on the nitrogen—as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be in crystalline form or either as free compounds or as solvates and it is intended that those forms are within the scope of the present invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

Any compound that is a prodrug of a compound of formula (I) is within the scope of the invention. The term “prodrug” is used in its broadest sense and encompasses those Derivatives that are converted in vivo to the compounds of the invention. Such Derivatives would readily occur to those skilled in the art, and include, depending on the functional groups present in the molecule and without limitation, the following Derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or ¹⁵N-enriched nitrogen are within the scope of this invention.

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I) or, or of its salts, solvates or prodrugs.

In another preferred embodiment of the combination of active substances according to the invention according to formula (I), at least one compound (A) or at least one compound (B) or at least one compound (A) and one compound (B) binding to the 5HT6-receptor is/are selected from sulfonamide compounds according to formula (Ia)

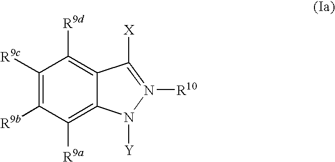

wherein
   either
   Y represents S(O₂)—R¹³, while X represents R¹;
   or
   X represents R¹, while Y represents (CH₂)ₙ—R¹¹ or
   Y represents R¹, while X represents (CH₂)ₙ—R¹²;
   while
   one of R^{9a}, R^{9b}, R^{9c}, or R^{9d} represents N(R³)—S(O₂)-A,
and wherein
A, R¹; R³, R^{9a}, R^{9b}, R^{9c}, R^{9d}, R¹⁰, R¹¹, R¹², R¹³ and n are as defined above.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (I) or (Ia)
wherein
=Z either represents ═C(R¹⁰) or —CH₂ or ═N;
and wherein
   either
Y represents —S(O₂)—R¹³, while X represents R¹;
   or
X represents R¹, while Y represents (CH₂), —R¹¹ or
Y represents R¹, while X represents (CH₂)ₙ—R¹²;
   while
   one of R^{9a}, R^{9b}, R^{9c}, or R^{9d} represents N(R³)—S(O₂)-A, or N—(S(O₂)-A)₂;
and wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—Cl—alkyl, —C(═O)—OH, —C(═O)—C₁₋₄-alkyl, C(═O)—O—C₁₋₆-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d}— if not N(R³)—S(O₂)-A or N—(S(O₂)-A)₂— independently from one another, each represent a hydrogen atom; —NO₂; —NH₂; —SH; —OH; —CN; —C(═O)—H; —C(═O)—O—C₁₋₅alkyl; —C(═O)—C₁₋₆-alkyl; —O—C₁₋₅-alkyl; —S—C₁₋₆-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH;
R¹⁰ represents a hydrogen atom; a linear or branched optionally at least mono-substituted C₁₋₅alkyl radical;
R¹¹ represents NR²R⁵ or a saturated or unsaturated 3-, 4,5-, 6,7- or 8-membered cycloaliphatic radical, which may be substituted with 1, 2 or 3 substituent(s) independently selected from the group consisting of C₁₋₅alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₄-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
R¹² represents R¹¹,
   or
   represents

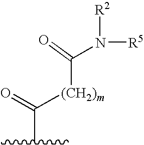

with n being 0;
or represents —C(OC₁₋₅-alkyl)-(CH₂)ₘ—R¹¹;
R¹³ represents a saturated or unsaturated, optionally at least mono-substituted C₅₋₇-cycloaliphatic radical, or —CHR′R″; with R′ and R″ identical or different, each representing a saturated or unsaturated, linear or branched, optionally at least mono-substituted C₁₋₅-alkyl radical;
n being 0, 1, 2, 3 or 4;
m being 0, 1, 2, 3 or 4;

R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —C—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
R² and R^{r} together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur.

In another preferred embodiment of the combination of active substances according to the invention according to formula (I), at least one compound (A) or at least one compound (B) or at least one compound (A) and one compound (B) binding to the 5HT6-receptor is/are selected from sulfonamide compounds according to formula (Ib) or (Ic)

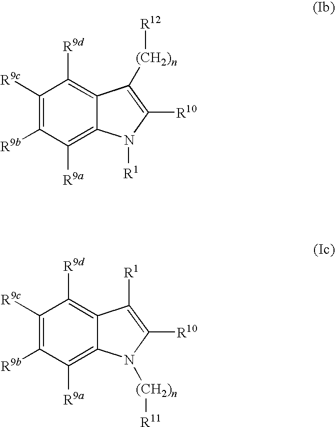

and wherein R¹, R^{9a}, R^{9b}, R^{9c}, R^{9d}, R¹⁰, R¹¹, R¹² and n have the meaning given above.

In another preferred embodiment of the combination of active substances according to the invention according to formula (Ib), at least one compound (A) or at least one compound (B) or at least one compound (A) and one compound (B) binding to the 5HT6-receptor is/are selected from sulfonamide compounds according to formulas (Iba), (Ibb), or (Ibc)

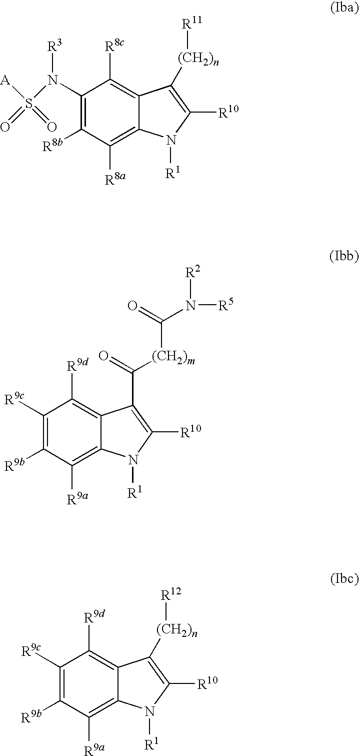

and wherein A, R¹, R², R³, R⁵, R^{9a}, R^{9b}, R^{9c}, R^{9d}, R¹⁰, R¹¹, R¹², m and n have the meaning given above and
wherein
   R^{8a}, R^{8b}, R^{8c} independently from one another, each represent a hydrogen atom; —NO₂; —NH₂; —SH; —OH; —CN; —C(═O)—H; —C(═O)—R′; —C(═O)—O—R′; —OR′; —SR′; —N(R′)-S(═O)₂—R′; —NH—R′; —NR′R″; F; Cl, Br; I; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or a 5- to 14-membered aryl or heteroaryl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—Cl s-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may be bonded via a linear or branched C₁₋₆ alkylene group and wherein the heteroaryl radical contains 1, 2 or 3 heteroatom(s) independently selected from the group consisting of nitrogen, oxygen and sulfur as ring member(s);
   preferably R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅alkyl; —O—C₁₋₅-alkyl; —S—C₁₋₅-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH.

In another preferred embodiment of the combination of active substances according to the invention according to formula (Ac), at least one compound (A) or at least one compound (B) or at least one compound (A) and one compound (B) binding to the 5HT6-receptor is/are selected from sulfonamide compounds according to formulas (Ica), (lcb), (Icc), or (Icd),

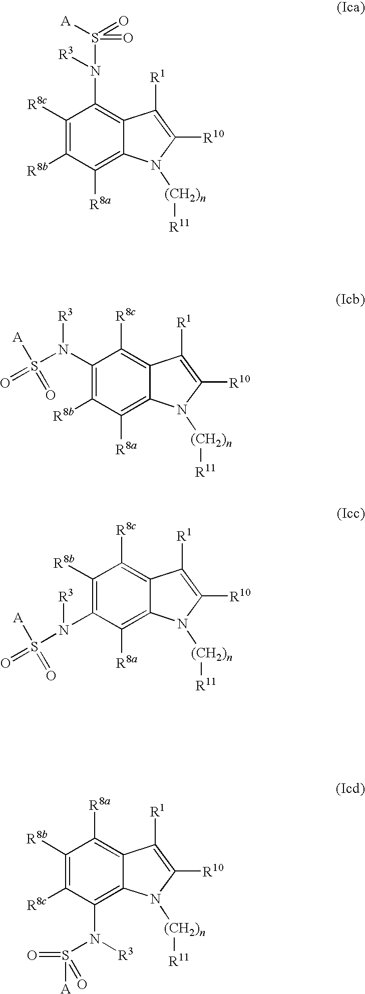

and wherein A, R¹, R³, R¹⁰, R¹¹, R¹², m and n have the meaning given above and wherein R^{8a}, R^{8b}, R^{8c} have the meaning given above.

In a very preferred embodiment of the combination of active substances according to the invention, at least one compound (A) or at least one compound (B) or at least one compound (A) and one compound (B) binding to the 5HT6-receptor is/are selected from sulfonamide compounds according to formula (Iba)

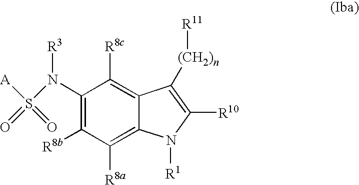

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—CH₅, —NO₂, —CN, —NH—CH₃ and —S—CH₃;
   R^{8a}, R^{8b}, R^{8c} each represent a hydrogen atom;
   R¹⁰ represents a hydrogen atom;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1,2, 3 or 4;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
      or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.
Compounds according to formula (Iba) are known from WO03/042175 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Iba) is/are selected from the following group of sulfonamide compounds consisting of:
[1] N-[3-(2-diethylaminoethyl)-1H-indol-5-yl]-5-chloro-3-methylbenzo[b]thiophene-2-sulphonamide.
[2] N-[3-(2-diethylaminoethyl)-1H-indol-5-yl]naphthalene-1-sulphonamide.
[3] Hydrochloride N-[3-(2-diethylaminoethyl)-1H-indol-5-yl]naphthalene-1-sulphonamide.
[4] N-[3-(2-diethylaminoethyl)-1H-indol-5-yl]-3,5-dichlorobenzenesulphonamide.
[5] N-[3-(2-diethylaminoethyl)-1H-indol-5-yl]-4-phenylbenzenesulphonamide.
[6] N-[3-(2-diethylaminoethyl)-1H-indol-5-yl]-5-chlorothiophene-2-sulphonamide.
[7] N-[3-(2-dimethylaminoethyl)-1H-indol-5-yl]-5-chloro-3-methylbenzo[b]thiophene-2-sulphonamide.
[8] N-[3-(2-dimethylaminoethyl)-1H-indol-5-yl]naphthalene-1-sulphonamide.
[9] N-[3-(2-dimethylamino-ethyl)-1H-indol-5-yl]-6-chloroimidazo[2,1-b]thiazol-5-sulphonamide.
[10] N-[3-(1-methylpiperidin-4-yl)-1H-indol-5-yl]-5-chloro-3-methylbenzo[b]thiophene-2-sulphonamide.
[11] N-[3-(1-methylpiperidin-4-yl)-1H-indol-5-yl]-5-chloro-3-methylbenzo[b]thiophene-2-sulphonamide hydrochloride.
[12] N-[3-(1-methylpiperidin-4-yl)-1H-indol-5-yl]naphthalene-1-sulphonamide.
[13] N-[3-(1-methylpiperidin-4-yl)-1H-indol-5-yl]naphthalene-1-sulphonamide hydrochloride.
[14] N-[3-(1-methylpiperidin-4-yl)-1H-indol-5-yl]-5-chlorothiophene-2-sulphonamide.
[15] N-[3-(1-methylpiperidin-4-yl)-1H-indol-5-yl]-4-phenylbenzenesulphonamide.
[16] N-[3-(1-methylpiperidin-4-yl)-1 indol-5-yl]quinoline-8-sulphonamide.
[17] N-[3-(2-diethylaminoethyl)-1H-indol-5-yl]naphthalene-2-sulphonamide.
[18] N-[3-(1-methyl-1,2,3,6-tetrahydropyridin-4-yl)-1H-indol-5-yl]naphthalene-1-sulphonamide.
[19] N-[3-(4-methylpiperazin-1-yl)methyl-1H-indol-5-yl]-5-chloro-3-methylbenzo[b]thiophene-2-sulphonamide.
[20] N-[3-(2-dimethylaminoethyl)-1H-indol-5-yl]-5-(2-pyridil)thiophene-2-sulphonamide.
[21] N-[3-(2-dimethylaminoethyl)-1H-indol-5-yl]-2,1,3-benzothiadiazol-4-sulphonamide.
[22] N-[3-(2-dimethylaminoethyl)-1H-indol-5-yl]quinoline-8-sulphonamide.
[23] N-[3-(2-dimethylaminoethyl)-1H-indol-5-yl]-5-chloronaphthalene-2-sulphonamide.
[24] N-[3-(2-dimethylaminoethyl)-1H-indol-5-yl]-4-phenoxybenzenesulphonamide.
[25] N-[3-(2-dimethylaminoethyl)-1H-indol-5-yl]-4-phenylbenzenesulphonamide.
[26] N-[3-(2-diethylaminoethyl)-1H-indol-5-yl]-N-ethyl-naphthalene-2-sulphonamide.
[27] N-{3-[2-(morpholin-4-yl)ethyl]-1H-indol-5-yl}-5-chloro-3-methylbenzo[b]thiophene-2-sulphonamide.
[28] N-{3-[2-(morpholin-4-yl)ethyl]-1H-indol-5-yl}naphthalene-1-sulphonamide.
[29] N-[3-(2-diethylaminoethyl)-1H-indol-5-yl]naphthalene-2-sulphonamide.
[30] N-[3-dimethylaminomethyl-1H-indol-5-yl]-5-chloro-3-methylbenzo[b]thiophene-2-sulphonamide.
[31] N-[3-(2-dipropylaminoethyl)-1H-indol-5-yl]naphthalene-1-sulphonamide.
[32] N-[3-(2-dipropylaminoethyl)-1H-indol-5-yl]-5-chloro-3-methylbenzo[b]thiophene-2-sulphonamide.
[33] N-[3-(2-dibutylaminoethyl)-1H-indol-5-yl]-5-chloro-3-methylbenzo[b]thiophene-2-sulphonamide.
[34] N-[3-(2-dibutylaminoethyl)-1H-indol-5-yl]naphthalene-1-sulphonamide.
[35] N-[3-(2-diethylaminoethyl)-1H-indol-5-yl]-5-chloronaphthalene-1-sulphonamide.
[36] N-[3-(2-diethylaminoethyl)-1H-indol-5-yl]-trans-β-styrenesulphonamide.
[37] N-[3-(4-methylpiperazin-1-yl)methyl-1H-indol-5-yl]-trans-β-styrenesulphonamide.
[38] N-[3-(octahydroindolizin-7-yl)-1H-indol-5-yl]-5-chloro-3-methylbenzo[b]thiophene-2-sulphonamide.
[39] N-[3-(2-diethylaminoethyl)-1H-indol-5-yl]-6-chloroimidazo[2,1-b]thiazol-5-sulphonamide.
[40] N-{3-[2-(morpholin-4-yl)ethyl]-1H-indol-5-yl}naphthalene-2-sulphonamide.
[41] N-[3-(4-methylpiperazin-1-yl)methyl-1H-indol-5-yl]-α-toluenesulphonamide.
[42] N-[3-(3-diethylaminopropyl)-1H-indol-5-yl]naphthalene-2-sulphonamide.
[43] N-[3-(3-diethylaminopropyl)-1H-indol-5-yl]-5-chloro-3-methylbenzo[b]thiophene-2-sulphonamide.
[44] N-{3-[2-(pyrrolidin-1-yl)ethyl]-1H-indol-5-yl}-5-chloro-3-methylbenzo[b]thiophene-2-sulphonamide.
[45] N-{3-[2-(pyrrolidin-1-yl)ethyl]-1H-indol-5-yl}naphthalene-1-sulphonamide.
[46] N-{3-[2-(pyrrolidin-1-yl)ethyl]-1H-indol-5-yl}naphthalene-2-sulphonamide.
[47] N-[3-(2-dipropylaminoethyl)-1H-indol-5-yl]naphthalene-2-sulphonamide.
[48] N-[3-(2-dimethylaminoethyl)-1H-indol-5-yl]-5-chloronaphthalene-1-sulphonamide.
[49] N-[3-(2-dimethylaminoethyl)-1H-indol-5-yl]naphthalene-2-sulphonamide.
[50] N-(3-[2-(morpholin-4-yl)ethyl]-1H-indol-5-yl)quinoline-8-sulphonamide.
[51] N-{3-[2-(morpholin-4-yl)ethyl]-1H-indol-5-yl}-4-phenylbenzenesulphonamide.
[52] N-[3-(4-methylpiperazin-1-yl)ethyl-1H-indol-5-yl]naphthalene-2-sulphonamide.
[53] N-[3-(4-methylpiperazin-1-yl)ethyl-1H-indol-5-yl]-5-chloronaphthalene-1-sulphonamide;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Ibb)

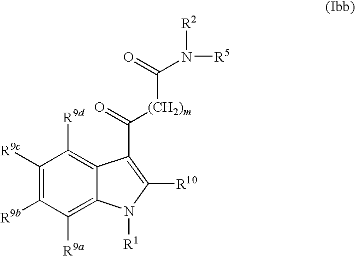

wherein
   one of R^{9a}, R^{9b}, R^{9c}, or R^{9d} represents N(R³)—S(O₂)-A, or N—(S(O₂)-A)₂;
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d}— if not N(R³)—S(O₂)-A or N—(S(O₂)-A)₂— independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH;
R¹⁰ represents a hydrogen atom; a linear or branched optionally at least mono-substituted C₁₋₅-alkyl radical;
m represents 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

Compounds according to formula (Ibb) are known from WO06/015867 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Ibb) is/are selected from the following group of sulfonamide compounds consisting of:
2-[5-(5-Chloro-3-methyl-benzo[b]thiophene-2-sulfonylamino)-1H-indol-3-yl]-N,N-diethyl-2-oxoacetamide.
N,N-Diethyl-2-[5-(naphthalene-2-sulfonylamino)-1H-indol-3-yl]-2-oxo-acetamide.
N,N-Diethyl-2-[5-(naphtalene-1-sulfonylamino)-1H-indol-3-yl]-2-oxo-acetamide.
2-[5-(Biphenyl-4-sulfonylamino)-1H-indol-3-yl]-N,N-diethyl-2-oxo-acetamide.
N,N-Diethyl-2-oxo-2-[5-(quinoline-8-sulfonylamino)-1H-indol-3-yl]-acetamide.
N,N-Dimethyl-2-[5-(naphthalene-2-sulfonylamino)-1H-indol-3-yl]-2-oxo-acetamide.
N,N-Dimethyl-2-[5-(naphtalene-1-sulfonylamino)-1H-indol-3-yl]-2-oxo-acetamide.
2-[5-(5-Chloro-3-methyl-benzo[b]thiophene-2-sulfonyl-amino)-1H-indol-3-yl]-N,N-dimethyl-2-oxo-acetamide.
2-[5-(6-Chloro-imidazo[2,1-b]thiazole-5-sulfonylamino)-1H-indol-3-yl]-N,N-diethyl-2-oxo-acetamide.
2-[5-(6-Chloro-imidazo[2,1-b]thiazole-5-sulfonylamino)-1H-indol-3-yl]-N,N-dimethyl-2-oxo-acetamide.
N,N-Dimethyl-2-[4-(naphthalene-1-sulfonylamino)-1H-indol-3-yl]-2-oxo-acetamide. 2-[4-(5-Chloro-3-methyl-benzo[b]thiophene-2-sulfonylamino)-1H-indol-3-yl]-N,N-dimethyl-2-oxo-acetamide.
2-[4-(6-Chloro-imidazo[2,1-b]thiazole-5-sulfonylamino)-1H-indol-3-yl]-N,N-dimethyl-2-oxo-acetamide.
N,N-Dimethyl-2-[5-[(4-fluoro-3-methyl-phenyl)-1-sulfonylamino]-1H-indol-3-yl]-2-oxo-acetamide.
5-(3-Dimethylaminooxalyl-1H-indol-5-ylsulfamoyl)-3-methyl-benzofuran-2-carboxylic acid ethyl ester.
2-[5-(Biphenyl-4-sulfonylamino)-1H-indol-3-yl]-N,N-dimethyl-2-oxo-acetamide.
N,N-Dimethyl-2-oxo-2-[5-(2-oxo-2,3-dihydro-benzoxazole-6-sulfonylamino)-1H-indol-3-yl]-acetamide.
N,N-Dimethyl-2-oxo-2-[5-(2-oxo-2,3-dihydrobenzo[d]thiazole-6-sulfonamido)-1H-indol-3-yl]acetamide.
2-[5-[(4-Cyclohexyl-phenyl)-1-sulfonylamino]-1H-indol-3-yl]-N,N-dimethyl-2-oxo-acetamide.
N,N-Dimethyl-2-[5-[(4-phenoxy-phenyl)-1-sulfonylamino]-1H-indol-3-yl]-2-oxo-acetamide.
2-(5-(5-chloro-3-methylbenzo[b]thiophene-2-sulfonamido)-2-methyl-1H-indol-3-yl)-N,N-dimethyl-2-oxoacetamide.
2-(5-(6-chloroimidazo[2,1-b]thiazole-5-sulfonamido)-2-methyl-1H-indol-3-yl)-N,N-dim ethyl-2-oxoacetamide.
2-(6(5-chloro-3-methylbenzo[b]thiophene-2-sulfonamido)-1H-indol-3-yl)-N,N-dimethyl-2-oxoacetamide.
N,N-dimethyl-2-(6-(naphthalene-3-sulfonamido)-1H-indo-3-yl)-2-oxoacetamide.
2-(6-(bipheny-4-sulfonamido)-1H-indol-3-yl)-N,N-dimethyl-2-oxoacetamide.
N,N-dimethyl-2-(6-(naphthalene-1-sulfonamido)-1H-indo-3-yl)-2-oxoacetamide.
N,N-dimethyl-2-(6-(2-(naphthalen-1-yl)ethylsulfonamido)-1H-indo-3-yl)-2-oxoacetamide.
N,N-dimethyl-2-oxo-2-(6-(4-phenoxyphenylsulfonamido)-1H-indol-3-yl)acetamide.
2-(6-(3,4-dichlorothiophene-2-sulfonamido)-1H-indol-3-yl)-N,N-dimethyl-2-oxoacetamide.
2-(6-(3,5-dichlorophenylsulonamido)-1H-indol-3-yl)-N,N-dimethyl-2-oxoacetamide.
2-(6-(1 chloronaphthalene-6-sulfonamido)-1H-indol-3-yl)-N,N-dimethyl-2-oxoacetamide.
2-(6-(6-chloroimidazo[2,1-b]thiazole-5-sulfonamido)-1H-indol-3-yl)-N,N-dimethyl-2-oxoacetamide.
N,N-diethyl-2-(2-methyl-5-(5-methyl-1-phenyl-1H-pyrazole-4-sulfonamido)-1H-indol-3-yl)-2-oxoacetamide.
N,N-diethyl-2-(2-methyl-5-(1,3,5-trimethyl-1H-pyrazolesulfonamido)-1H-indol-3-yl)-2-oxoacetamide;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Ibc)

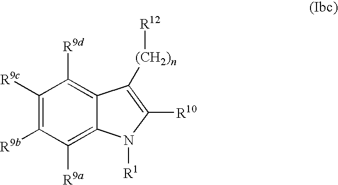

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents hydrogen;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³— if present—represents hydrogen;
   R^{9a}, R^{9b}, R^{9c}, R^{9d}— if not N(R³)—S(O₂)-A or N—(S(O₂)-A)₂— independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅alkyl radical which may be substituted with 1, 2 or 3 substituent(s) independently selected from the group consisting of F, Cl, Br, —OH, —NH₂, and —SH; with the proviso that one of R^{9a}, R^{9b}, R^{9c}, or R^{9d} represents N(R³)—S(O₂)-A, or N—(S(O₂)-A)₂;
   R¹⁰ represents a hydrogen atom; a linear or branched optionally at least mono-substituted C₁₋₅-alkyl radical; preferably R¹⁰ represents hydrogen;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   R¹² represents R¹¹, or represents —C(OC₁₋₅-alkyl)-(CH₂)ₘ—R¹¹;
   n being 0, 1, 2, 3 or 4;
   m being 0, 1, 2, or 3;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
      or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.
Compounds according to formula (Ibc) are known from WO06/024535 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Ibc) is/are selected from the following group of sulfonamide compounds consisting of:
5-chloro-N-(3-(2-(diethylamino)ethyl)-1H-indol-6-yl)-3-methylbenzo[b]thiophene-2-sulfonamide
N-(3-(2-(diethylamino)ethyl)-1H-indol-6-yl)naphthalene-2-sulfonamide
N-(3-(2-(diethylamino)ethyl)-1H-indol-6-yl)naphthalene-1-sulfonamide
6-chloro-N-(3-(2-(diethylamino)ethyl)-1H-indol-6-yl)imidazo[2,1-b]thiazole-5-sulfonamide
N-(3-(2-(diethylamino)ethyl)-1H-indol-6-yl)-4-phenylbenzenesulfonamide
N-(3-(2-(diethylamino)ethyl)-1H-indol-6-yl)-4-phenoxybenzenesulfonamide
3,5-dichloro-N-(3-(2-(diethylamino)ethyl)-1H-indol-6-yl)benzenesulfonamide
4,5-dichloro-N-(3-(2-(diethylamino)ethyl)-1H-indol-6-yl)thiophene-2-sulfonamide
5-chloro-N-(3-(2-(diethylamino)ethyl)-1H-indol-6-yl)naphthalene-1-sulfonamide
5-chloro-N-(3-(2-(dimethylamino)ethyl)-1H-indol-6-yl)-3-methylbenzo[b]thiophene-2-sulfonamide
N-(3-(2-(dimethylamino)ethyl)-1H-indol-6-yl)naphthalene-2-sulfonamide
N-(3-(2-(dimethylamino)ethyl)-1H-indol-6-yl)naphthalene-1-sulfonamide
6-chloro-N-(3-(2-(dimethylamino)ethyl)-1H-indol-6-yl)imidazo[2,1-b]thiazole-5-sulfonamide
N-(3-(2-(dimethylamino)ethyl)-1H-indol-6-yl)-4-phenylbenzenesulfonamide
N-(3-(2-(dimethylamino)ethyl)-1H-indol-6-yl)-2-(naphthalen-1-yl)ethanesulfonamide
N-(3-(2-(dimethylamino)ethyl)-1H-indol-6-yl)-4-phenoxybenzenesulfonamide
3,5-dichloro-N-(3-(2-(dimethylamino)ethyl)-1H-indol-6-yl)benzenesulfonamide
4,5-dichloro-N-(3-(2-(dimethylamino)ethyl)-1H-indol-6-yl)thiophene-2-sulfonamide
5-chloro-N-(3-(2-(dimethylamino)ethyl)-1H-indol-6-yl)naphthalene-1-sulfonamideo
5-chloro-N-(3-(2-(dimethylamino)-1-ethoxyethyl)-1H-indol-7-yl)-3-methylbenzo[b]thiophene-2-sulfonamide
5-chloro-N-(3-(2-(dimethylamino)ethyl)-1H-indol-7-yl)-3-methylbenzo[b]thiophene-2-sulfonamide
7-bis(5-chloro-3-methylbenzo[b]thiophene-2-sulfonyl)amino-3-(2-(diethylamino)-1-ethoxyethyl)-1H-indole
5-chloro-N-(3-(2-(diethylamino)-1-ethoxyethyl)-1H-indol-7-yl)-3-methylbenzo[b]thiophene-2-sulfonamide
7-bis(5-chloro-3-methylbenzo[b]thiophene-2-sulfonyl)amino-3-(2-(dimethylamino)ethyl)-1H-indole
7-bis(5-chloro-3-mathylbenzo[b]thiophene-2-sulfonyl)amino-3-(2-(diethylamino)ethyl)-1H-indole
5-chloro-N-(3-(2-(diethylamino)ethyl)-1H-indol-7-yl)-3-methylbenzo[b]thiophene-2-sulfonamide
7-bis(6-chloroimidazo[2,1-b]thiazol-5-ylsulfonyl)amino-3-(2-(dimethylamino)ethyl)-1H-indole
N-(3-(2-(dimethylamino)ethyl)-1H-indol-4-yl)-4-biphenylsulfonamide
N-(3-(2-(dimethylamino)ethyl)-1H-indol-4-yl)-4-phenoxybenzenesulfonamide
3,5-dichloro-N-(3-(2-(dimethylamino)ethyl)-1H-indol-4-yl)benzenesulfonamide
5-chloro-N-(3-(2-(dimethylamino)ethyl)-1H-indol-4-yl)-3-methylbenzo[b]thiophene-2-sulfonamide
N-(3-(2-(dimethylamino)ethyl)-1H-indol-4-yl)naphthalene-1-sulfonamide
5-chloro-N-(3-(2-(dimethylamino)ethyl)-1H-indol-4-yl)naphthalene-2-sulfonamide
N-(3-(2-(dimethylamino)ethyl)-1H-indol-4-yl)naphthalene-2-sulfonamide
6-chloro-N-(3-(2-(dimethylamino)ethyl)-1H-indol-4-yl)imidazo[2,1-b]thiazole-5-sulfonamide
N-(3-(2-(dimethylamino)ethyl)-1H-indol-4-yl)-2-(naphthalen-1-yl)ethanesulfonamide
6-bis(6-chloroimidazo[2,1-b]thiazol-5-ylsulfonyl)amino-3-(2-(dimethylamino)ethyl)-1H-indole
6-bis(3,5-dichlorobenzenesulfonyl)amino-3-(2-(dimethylamino)ethyl)-1H-indole
6-bis(4,5-dichlorothiophene-2-sulfonyl)amino-3-(2-(dimethylamino)ethyl)-1H-indole
6-bis(5-chloro-3-methylbenzo[b]thiophene-2-sulfonyl)amino-3-(2-(dimethylamino)-1-ethoxyethyl)-1H-indole
N-(3-(2-(diethylamino)ethyl)-7-methoxy-1H-indol-5-yl)naphthalene-2-sulfonamide
N-(3-(2-(diethylamino)ethyl)-7-methoxy-1H-indol-5-yl)benzo[c][1,2,5]thiadiazole-4-sulfonamide
6-chloro-N-(3-(2-(diethylamino)ethyl)-7-methoxy-1H-indol-5-yl)imidazo[2,1-b]thiazole-5-sulfonamide
ethyl 6-(5-chloro-3-methylbenzo[b]thiophene-2-sulfonamido)-3-(1-methylpiperidin-4-yl)-1H-indole-5-carboxylate
N-(5-bromo-3-(2-(pyrrolidin-1-yl)ethyl)-1H-indol-7-yl)-6-chloroimidazo[2,1-b]thiazole-5-sulfonamide
N-(4-bromo-3-(1-methylpiperidin-4-yl)-1H-indol-6-yl)naphthalene-1-sulfonamide
N-(7-bromo-3-(2-(dimethylamino)ethyl)-1H-indol-5-yl)benzofuran-2-sulfonamide
N-(7-methoxy-3-(2-(pyrrolidin-1-yl)ethyl)-1H-indol-5-yl)benzo[c][1,2,5]thiadiazole-4-sulfonamide
N-(7-methoxy-3-(2-(pyrrolidin-1-yl)ethyl)-1H-indol-5-yl)naphthalene-2-sulfonamide
6-chloro-N-(7-methoxy-3-(2-(pyrrolidin-1-yl)ethyl)-1H-indol-5-yl)imidazo[2,1-b]thiazole-5-sulfonamide;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively;
   preferably is selected from:
N-(3-(2-(diethylamino)ethyl)-7-methoxy-1H-indol-5-yl)naphthalene-2-sulfonamide,
N-(3-(2-(diethylamino)ethyl)-7-methoxy-1H-indol-5-yl)benzo[c][1,2,5]thiadiazole-4-sulfonamide,
6-chloro-N-(3-(2-(diethylamino)ethyl)-7-methoxy-1H-indol-5-yl)imidazo[2,1-b]thiazole-5-sulfonamide,
ethyl 6-(5-chloro-3-methylbenzo[b]thiophene-2-sulfonamido)-3-(1-methylpiperidin-4-50 yl)-1H-indole-5-carboxylate,
N-(5-bromo-3-(2-(pyrrolidin-1-yl)ethyl)-1H-indol-7-yl)-6-chloroimidazo[2,1-b]thiazole-5-sulfonamide,
N-(4-bromo-3-(1-methylpiperidin-4-yl)-1H-indol-6-yl)naphthalene-1-sulfonamide,
N-(7-bromo-3-(2-(dimethylamino)ethyl)-1H-indol-5-yl)benzofuran-2-sulfonamide,
N-(7-methoxy-3-(2-(pyrrolidin-1-yl)ethyl)-1H-indol-5-yl)benzo[c][1,2,5]thiadiazole-4-sulfonamide,
N-(7-methoxy-3-(2-(pyrrolidin-1-yl)ethyl)-1H-indol-5-yl)naphthalene-2-sulfonamide and
6-chloro-N-(7-methoxy-3-(2-(pyrrolidin-1-yl)ethyl)-1H-indol-5-yl)imidazo[2,1-b]thiazole-5-sulfonamide.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Ica)

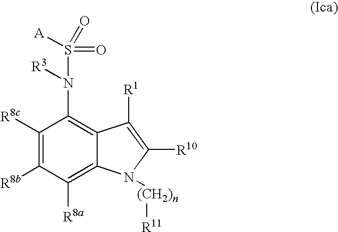

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
   R^{8a}, R^{8b}, R^{8c} independently from one another, each represent a hydrogen atom; —NO₂; —NH₂; —SH; —OH; —CN; —C(═O)—H; —C(═O)—O—C₁₋₈-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
   R¹⁰ represents a hydrogen atom; a linear or branched optionally at least mono-substituted C₁₋₅-alkyl radical; preferably R¹⁰ represents a hydrogen atom;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4; preferably n being 2;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.
Compounds according to formula (Ica) are known from WO05/013978 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Ica) is/are selected from the following group of sulfonamide compounds consisting of:
[1] N-[1-(2-dimethylaminoethyl)-1H-indole-4-yl]-5-chloro-3-methylbenzo[b]thiophene-2-sulfonamide,
[2] N-[1-(2-dimethylaminoethyl)-1H-indole-4-yl]-naphtalene-2-sulfonamide,
[3] N-[1-(2-dimethylaminoethyl)-1H-indole-4-yl]-naphtalene-1-sulfonamide,
[4] N-[1-(2-dimethylaminoethyl)-1H-indole-4-yl]-4-phenylbenzenesulfonamide,
[5] N-[1-(2-dimethylaminoethyl)-1H-indole-4-yl]-2-(naphtalene-1-yl)-ethanesulfonamide,
[6] N-[1-(2-dimethylaminoethyl)-1H-indole-4-yl]-4-phenoxybenzenesulonamide,
[7] N-[1-(2-dimethylaminoethyl)-1H-indole-4-yl]-3,5-dichlorobenzenesulfonamide and
[8] 6-chloro-N-[1-(2-dimethylaminoethyl)-1H-indol-4-yl]-imidazo[2,1-b]thiazole-5-sulfonamide
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Icb)

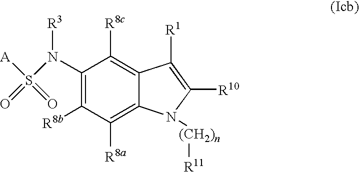

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁ s-alkyl, —C(═O)—OH, —C(═O)—C₁₋₆-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R³ represents a hydrogen atom;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
   R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH; preferably R^{8a}, R^{8b}, R^{8c}, each represent a hydrogen atom;
   R¹⁰ represents a hydrogen atom; a linear or branched optionally at least mono-substituted C₁₋₅-alkyl radical; preferably R¹⁰ represents a hydrogen atom or methyl;
R¹¹ represents NR²R⁵ or a saturated or unsaturated 3-, 4-, 5-, 6,7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4; preferably n being 2;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.
Compounds according to formula (Icb) are known from WO05/013977 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Icb) is/are selected from the following group of sulfonamide compounds consisting of:
[1] N-[1-(2-dimethylaminoethyl)-1H-indole-5-yl]-5-chloro-3-methylbenzo[b]thiophene-2-sulfonamide,
[2] N-[1-(2-dimethylaminoethyl)-1H-indole-5-yl]-naphthalene-2-sulfonamide,
[3] N-[1-(2-dimethylaminoethyl)-1H-indole-5-yl]-naphthalene-1-sulfonamide,
[4] N-[1-(2-dimethylaminoethyl)-1H-indole-5-yl]-5-chloronaphthalene-1-sulfonamide,
[5] N-[1-(2-dimethylaminoethyl)-1H-indole-5-yl]-benzenesulfonamide,
[6] N-[1-(2-dimethylaminoethyl)-1H-indole-5-yl]-quinoline-8-sulfonamide,
[7] N-[1-(2-dimethylaminoethyl)-1H-indole-5-yl]-4-phenoxybenzenesulfonamide,
[8] N-[1-(2-dimethylaminoethyl)-1H-indole-5-yl]-4-methylbenzenesulfonamide,
[9] N-[1-(2-dimethylaminoethyl)-1H-indole-5-yl]-5-chlorothiophene-2-sulfonamide,
[10] N-[1-(2-dimethylaminoethyl)-1H-indole-5-yl]-benzo[1,2,5]thiadiazole-4-sulfonamide,
[11] N-[1-(2-dimethylaminoethyl)-1H-indole-5-yl]-6-chloroimidazo[2,1-b]thiazole-5-sulfonamide,
[12] N-[1-(2-dimethylaminoethyl)-1H-indole-5-yl]-3,5-dichlorobenzenesulfonamide,
[13] N-[1-(2-dimethylaminoethyl)-1H-indole-5-yl]-3-bromobenzenesulfonamide,
[14] N-[1-(2-dimethylaminoethyl)-1H-indole-5-yl]-3-nitrobenzenesulfonamide,
[15] N-[1-(2-dimethylaminoethyl)-1H-indole-5-yl]-1-phenylmethanesulfonamide,
[16] N-[1-(2-pyrrolidine-1-yl-ethyl)-1H-indole-5-yl]-naphthalene-2-sulfonamide,
[17] N-[1-(2-pyrrolidine-1-yl-ethyl)-1H-indole-5-yl]-naphthalene-1-sulfonamide,
[18] N-[1-(2-pyrrolidine-1-yl-ethyl)-1H-indole-5-yl]-5-chloro-3-methylbenzo[b]thiophene-2-sulfonamide,
[19] trans-N-[1-(2-dimethylaminoethyl)-1H-indole-5-yl]-2-phenylethenesulfonamide,
[20] N-[1-(2-dimethylaminoethyl)-1H-indole-5-yl]-4,5-dichlorothiophene-2-sulfonamide,
[21] N-[1-(2-dimethylaminoethyl)-1H-indole-5-yl]-4-acetylbenzenesulfonamide,
[22] N-[1-(2-dimethylaminoethyl)-1H-indole-5-yl]-4-bromobenzenesulfonamide,
[23] N-[1-(2-dimethylaminoethyl)-1H-indole-5-yl]-4-methoxybenzenesulfonamide,
[24] N-[3-(2-diethylaminoethyl)-1H-indole-5-yl]-5-chloro-3-methylbenzo[b]thiophene-2-sulfonamide,
[25] N-[1-(2-dimethylaminoethyl)-1H-indole-5-yl]-4-nitrobenzenesulfonamide,
[26] N-[1-(2-dimethylaminoethyl)-1H-indole-5-yl]-4-fluorobenzenesulfonamide,
[27] N-[1-(2-diethylaminoethyl)-1H-indole-5-yl]-6-chloroimidazo[2,1-b]thiazole-5-sulfonamide,
[28] N-[1-(2-pyrrolidine-1-yl-ethyl)-1H-indole-5-yl]-]-6-chloroimidazo[2,1-b]thiazole-5-sulfonamide,
[29] N-(1-(2-(diethylamino)ethyl)-1H-indol-5-yl)-naphthalene-2-sulfonamide,
[30] N-(1-(2-(diethylamino)ethyl)-1H-indol-5-yl)-naphthalene-1-sulonamide,
[31] N-(1-(2-(diethylamino)ethyl)-1H-indol-5-yl)-4-phenylbenzenesulfonamide,
[32] 5-chloro-N-(1-(2-(dimethylamino)ethyl)-2-methyl-1H-indol-5-yl)-3-methylbenzo[b]thiophene-2-sulfonamide,
[33] N-(1-(2-(dimethylamino)ethyl)-2-methyl-1H-indol-5-yl)-naphthalene-2-sulfonamide,
[34] N-(1-(2-(dimethylamino)ethyl)-2-methyl-1H-indol-5-yl)-naphthalene-1-sulfonamide,
[35] 6-chloro-N-(1-(2-(dimethylamino)ethyl)-2-methyl-1H-indol-5-yl)imidazo[2,1-b]thiazole-5-sulfonamide,
[36] N-(1-(2-(dimethylamino)ethyl)-2-methyl-1H-indol-5-yl)-4-phenylbenzenesulfonamide,
[37] N-(1-(2-dimethylamino)ethyl)-2-methyl-1H-indol-5-yl)-2-(naphth-1-yl)-ethanesulfonamide,
[38] N-(1-(2-(dimethylamino)ethyl)-2-methyl-1H-indol-5-yl)-4-phenoxy-benzenesulfonamide,
[39] 3,5-dichloro-N-(1-(2-(dimethylamino)ethyl)-2-methyl-1H-indol-5-yl)-benzenesulfonamide,
[40] N-(1-(2-(dimethylamino)ethyl)-2-methyl-1H-indol-5-yl)benzo[b]thiophene-3-sulfonamide,
[41] N-(1-(2-(diethylamino)ethyl)-1H-indol-5-yl)benzo[b]thiophene-3-sulfonamide and
[42] N-(1-(2-(dimethylamino)ethyl)-1H-indol-5-yl)benzo[b]thiophene-3-sulfonamide,
[43] 5-chloro-3-methyl-N-(1-(3-(piperidin-1-yl)propyl)-1H-indol-5-yl)benzo[b]thiophene-2-sulfonamide,
[44] N-(1-(3-(piperidin-1-yl)propyl)-1H-indol-5-yl)naphthalene-2-sulfonamide,
[45] N-(1-(3-(piperidin-1-yl)propyl)-1H-indol-5-yl)naphthalene-1-sulfonamide,
[46] 6-chloro-N-(1-(3-piperidin-1-yl)propyl)-1H-indol-5-yl)imidazo[2,1-b]thiazole-5-sulfonamide,
[47] 4-phenyl-N-(1-(3-(piperidin-1-yl)propyl)-1H-indol-5-yl)benzenesulfonamide,
[48] 2-(naphth-1-yl)-N-(1-(3-(piperidin-1-yl)propyl)-1H-indol-5-yl)ethanesulfonamide,
[49] 4-phenoxy-N-(1-(3-(piperidin-1-yl)propyl)-1H-indol-5-yl)benzenesulfonamide,
[50] 3,5-dichloro-N-(1-(3-(piperidin-1-yl)propyl)-1H-indol-5-yl)benzenesulfonylamide,
[51] 4,5-dichloro-N-(1-(3-(piperidin-1-yl)propyl)-1H-indol-5-yl)thiophene-2-sulfonamide and
[52] 5-chloro-N-(1-(3-(piperidin-1-yl)propyl)-1H-indol-5-yl)naphthalene-1-sulfonamide,
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Icc)

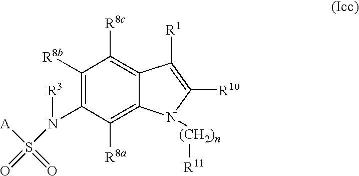

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅-alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
   R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
   R¹⁰ represents a hydrogen atom; a linear or branched optionally at least mono-substituted C₁₋₅-alkyl radical; preferably R¹⁰ represents a hydrogen atom;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4; preferably n being 2;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —OCH₃, —OCH₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
   R² and R⁶ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₆-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.
Compounds according to formula (Icc) are known from WO05/013976 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Icc) is/are selected from the following group of sulfonamide compounds consisting of:
[1] N-[1-(2-Dimethylaminoethyl)-1H-indol-6-yl]-5-chloro-3-methylbenzo[b]thiophene-2-sulfonamide,
[2] N-[1-(2-Dimethylaminoethyl)-1H-indol-6-yl]-naphthalene-2-sulfonamide,
[3] N-[1-(2-Dimethylaminoethyl)-1H-indol-6-yl]-naphthalene-1-sulfonamide,
[4] N-[1-(2-Dimethylaminoethyl)-1H-indol-6-yl]-6-chloroimidazo[2,1-b]thiazole-5-sulfonamide,
[5] N-[1-(2-Dimethylaminoethyl)-1H-indol-6-yl]-4-phenylbenzenesulfonamide,
[6] N-[1-(2-Dimethylaminoethyl)-1H-indol-6-yl]-2-(naphthalene-1-yl)-ethanesulfonamide,
[7] N-[1-(2-Dimethylaminoethyl)-1H-indol-6-yl]-4-phenoxybenzenesulfonamide,
[8] N-[1-(2-Dimethylaminoethyl)-1H-indol-6-yl]-3,5-dichlorobenzenesulfonamide,
[9] 5-Chloro-3-methyl-N-[1-[2-(pyrrolidin-1-yl)ethyl-1H-indol-6-yl]-benzo[b]thiophene-2-sulfonamide,
[10] N-(1-[2-(Pyrrolidin-1-yl)ethyl]-1H-indol-6-yl]-napthalene-2-sulfonamide,
[11] N-[1-[2-Pyrrolidin-1-yl]ethyl]-1H-indol-6-yl]-naphthalene-1-sulfonamide,
[12] 6-Chloro-N-[1-[2-(pyrrolidin-1-yl)ethyl]-1H-indol-6-yl]-imidazo[2,1-b]thiazole-5-sulfonamide,
[13] 4-Phenyl-N-(1-(2-(pyrrolidin-1-yl)ethyl)-1H-indol-6-yl)-benzenesulfonamide
[14] 2-(Naphthyl-1-yl)-N-(1-(2-(pyrrolidin-1-yl)ethyl)-1H-indol-6-yl)-ethansulfonamide,
[15] 4-Phenoxy-N-(1-(2-(pyrrolidin-1-yl)ethyl)-1H-indol-6-yl)-benzenesulfonamide and
[16] 3,5-Dichloro-N-(1-(2-(pyrrolidin-1-yl)-1H-indol-6-yl)-benzenesulfonamide,
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Icd)

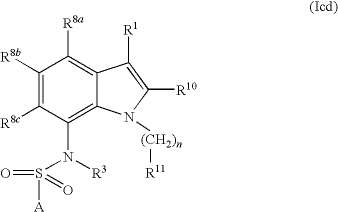

wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₄ alkyl, C(═O)—O—C₁₋₅alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂HF, —NO₂, —CN, —NH—CH₃ and —S—CH₃; preferably R³ represents a hydrogen atom;
   R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
   R¹⁰ represents a hydrogen atom; a linear or branched optionally at least mono-substituted C₁₋₅-alkyl radical; preferably R¹⁰ represents a hydrogen atom;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4; preferably n being 2;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₄-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

Compounds according to formula (Icd) are known from WO05/013979 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Icd) is/are selected from the following group of sulfonamide compounds consisting of:
[1] N-[1-(2-dimethylaminoethyl)-1H-indole-7-yl]-naphtalene-1-sulfonamide,
[2] N-[1-(2-dimethylaminoethyl)-1H-indole-7-yl]-5-chloro-3-methylbenzo[b]thiophene-2-sulfonamide,
[3] N-[1-(2-dimethylaminoethyl)-1H-indole-7-yl]-4-phenylbenzenesulfonamide and
[4] N-[1-(2-dimethylaminoethyl)-1H-indole-7-yl]-6-chloroimidazo[2,1-b]thiazole-5-sulfonamide
[5] 5-chloro-3-methyl-N-(1-(2-(pyrrolidin-1-yl)ethyl)-1H-indol-7-yl)-benzo[b]thiophen-2-sulfonamide,
[6] N-(1-(2-(pyrrolidin-1-yl)ethyl)-1H-indol-7-yl)naphthalene-1-sulfonamide,
[7] 6-chloro-N-(1-(2-(pyrroldin-1-yl)ethyl)-1H-indol-7-yl)imidazo[2,1-b]thiazole-5-sulfonamide and
[8] 2-(naphth-1-yl)-N-(1-(2-(pyrrolidin-1-yl)ethyl)-1H-indol-7-yl)ethansudonamide
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Id)

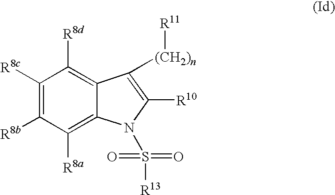

wherein
   R^{8a}, R^{8b}, R^{8c}, R^{8d} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH;
   R¹⁰ represents a hydrogen atom; a linear or branched optionally at least mono-substituted C₁₋₅-alkyl radical; preferably R² represents H;
   R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4;
   R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —OCH₃, —O—C₂H₅, —NO₂, —CN, —NH—CH₃ and —S—CH₃; optionally at least monosubstituted alkyl-aryl; or C(O)—R with R being an optionally at least mono-substituted aryl,
   R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
   or
   R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₄-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively
Compounds according to formula (Id) are known from WO05/013974 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Id) is/are selected from the following group of sulfonamide compounds consisting of:
[1] 1-Cyclohexanesulfonyl-3-(1-methyl-1,2,3,6-tetrahydropyridine-4-yl)-5-nitro-1H-indole,
[2] 5-Chloro-1-cyclohexanesulfonyl-3-(1-methyl-1,2,3,6-tetrahydropyridine-4-yl)-1H-indole,
[3] 5-Amino-1-cyclohexanesulfonyl-3-(1-methyl-1,2,3,6-tetrahydropyridine-4-yl)-1H-indole and
[4] 1-Cyclohexanesulfonyl-5-fluoro-3-(1,2,3,5,8,8a-hexahydro-indolizine-7-yl)-1H-indole hydrochloride;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from sulfonamide compounds according to formula (Ie)

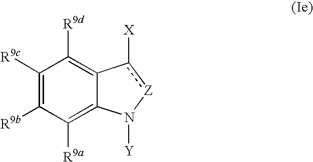

wherein
   wherein
   =Z either represents —CH₂ or ═N;
   and wherein
   X represents R¹, while Y represents (CH₂)ₙ—R¹¹ or
   Y represents R¹, while X represents (CH₂)ₙ—R¹¹;
      while
      one of R^{9a}, R^{9b}, R^{9c}, or R^{9d} represents N(R³)—S(O₂)-A;
   and wherein
   A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
   R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
   R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
   R^{9a}, R^{9b}, R^{9c}, R^{9d} —H not N(R³)—S(O₂)-A—independently from one another, each represent a hydrogen atom; —NO₂; —NH₂; —SH; —OH; —CN; —C(═O)—H; —C(═O)—O—C₁₋₅-alkyl; —C(═O)-C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{9a}, R^{9b}, R^{9c}, R^{9d}— if not N(R³)—S(O₂)-A—each represent a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
   n being 0, 1, 2, 3 or 4;
      R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —OC₂H₅, —NO₂, —CN, —NH—CH₃ and —S—CH₃; optionally at least monosubstituted alkyl-aryl; or C(O)—R with R being an optionally at least mono-substituted aryl,
      R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
      or
      R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅ alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
      optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

Compounds according to formula (Ie) are known from WO06/069809 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (Ie) is/are selected from the following group of sulfonamide compounds consisting of:
[1] N-(1-(2-(Dimethylamino)ethyl)-1H-indazol-6-yl)napthalene-2-sulphonamide;
[2] 5-Chloro-N-(1-(2-(dimethylamino)ethyl)-1H-indazol-6-yl)-3-methylbenzo[b]thiophene-2-sulfonamide;
[3] Naphthalene-2-sulonic acid [3-(1-methyl-piperidin-4-yl)-1H-indazol-5-yl]-amide,
[4] 5-Chloro-3-methyl-benzo[b]thiophene-2-sulfonic acid [3-(1-methyl-piperidin-4-yl)-1H-indazol-5-yl]-amide,
[5] Naphthalene-1-sulfonic acid [3-(1-methyl-piperidin-4-yl)-1H-indazol-5-yl]-amide,
[6] 4-Phenylbenzene-4-sulfonic acid [3-(1-methyl-piperidin-4-yl)-1H-indazol-5-yl]-amide,
[7] N-[3-(1-Methyl-piperidin-4-yl)-1H-indazol-5-yl]-4-phenoxy-benzenesulfonamide
[8] N-[3-(1-Methyl-piperidin-4-yl)-1H-indazol-5-yl]-benzenesulfonamide;
[9] N-[1-(2-Dimethylamino)ethyl)-2,3-dihydro-1H-indol-6-yl]-6-chloro-imidazo[2,1-b]thiazol-5-sulfonamide;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from compounds according to formula (II)

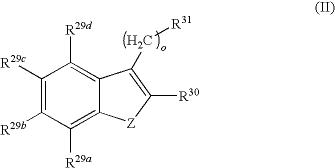

wherein
o is 0, 1, 2, 3 or 4
R³¹ represents a saturated or unsaturated cycloaliphatic radical, optionally at least monosubstituted, optionally at least with one heteroatom selected from N, O and S as a member of the ring that may be condensed with a mono or polycyclic annular system optionally at least monosubstituted; a —NR⁸R⁹ radical; a —ONR⁸R⁹ radical; —COOH; or —OH
where
R⁸ and R⁹ represent, independently of each other, a hydrogen atom; or a linear or branched, saturated or unsaturated C₁₋₅ aliphatic radical that may be substituted by 1, 2, 3 substituents selected independently from F, Cl, Br, —OH, —NH₂, —SH, —O—CH₃, —O—C₂H₅, —NO₂, —CN, —NH—CH₃ and —S—CH₃; or R⁸ and R⁹ together with nitrogen form a saturated, unsaturated or aromatic heterocyclic ring with 3 to 9 members, which may be substituted by 1, 2 or 3 substituents selected independently from C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₁₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl and which may contain 1, 2 or 3 additional heteroatoms independently selected from N, O and S as members of the ring
R^{29a}, R^{29b}, R^{29c} and R^{29d} represent, independently of one another, a hydrogen atom; —NO₂; —NH₂; —SH; —OH; —CN; —C(═O)—H; —C(═O)—R³¹; —OR³²; —SR³³; —SOR³⁴, —S(O)₂—R., —S(O)₂—N(R³)R³⁶, —N(R³⁷)—S(O)₂—R³⁸; —NH—R³⁹; —NR⁴⁰R⁴¹; —N(R⁴²—CO—R⁴³; F; Cl, Br; I; a linear or branched, saturated o unsaturated C₁-C₆ aliphatic radical, which may be substituted by 1, 2 or 3 substituents independently selected from F, Cl, Br, —OH, —NH₂, —SH, —O—CH₃, —O—C₂H₅, —NO₂, —CN, —NH—CH₃ and —S—CH₃; or an aryl or heteroaryl radical of 5 to 14 members, which may be substituted by 1, 2 or 3 substituents independently selected from —CF₃, C₁₋₅-alkyl, —O—C₁₋₅-alkyl, —S—C₁₋₅-alkyl, —C(O)—OH, —C(O)—O—C₁₋₅-alkyl, —O—C(O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(O)—C₁₋₅-alkyl, —N(C₁₋₅alkyl)-C(O)—C₁₋₅-alkyl, —NO₂, —CHO, —CF₂H, —CFH₂, —C(O)—NH₂, —C(O)—NH(C₁₋₅-alkyl), —C(O)—N(C₁₋₅-alkyl)₂, —S(O)₂—C₁₋₅-alkyl, —S(O)₂-phenyl, cyclopropyl, cyclobutyl, cyclopentyl, cyclohexyl, phenyl, phenoxy, benzyloxy and benzyl and which may be bonded by a linear or branched C₁-C₆ alkylene group, and where the heteroaryl radical contains 1, 2 or 3 heteroatoms independently selected from N, O and S as members of the ring; with the condition that at least one of the substituents R^{29a}, R^{29b}, R^{29c} and R^{29d} represents a —NO₂, —SOR³⁴, —S(O)₂—R³⁴, —S(O)₂—N(R³⁵)R³⁵, —N(R³⁷)—S(O)₂—R³, —N(R⁴²)—CO—R⁴³ radical;
Z represents:

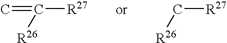

which respectively means (IIx) and (IIy) type compounds:

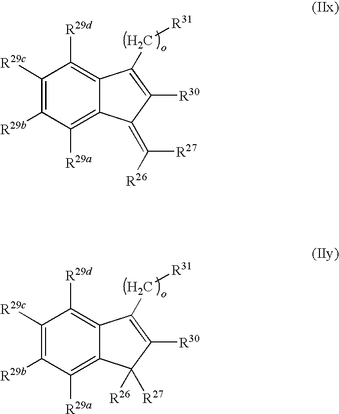

R²⁶ and R²⁷, identical or different, represent a hydrogen atom; NO₂; —NH₂; —SH; —OH; —CN; —C(═O)—R¹⁰; —OR¹¹; —SR¹²; F; Cl, Br; I; a linear or branched, saturated or unsaturated C₁-C₁₀ aliphatic radical, which may be substituted with 1, 2 or 3 substituents independently selected among F, Cl, Br, —OH, —SH, —O—CH₃, —O—C₂H₅, —NO₂, —CN and —S—CH₃; or an aryl or heteroaryl radical of 5 to 14 members, which may be substituted by 1, 2 or 3 substituents independently selected from —CF₃, C₁₋₅-alkyl, —O—C₁₋₅-alkyl, —S—C₁₋₆-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy, benzyloxy and benzyl and which may be bonded by a linear or branched C₁-C₆ alkylene, C₂-C₆ alkenylene or C₁-C₆ ylidene group, and where the heteroaryl radical contains 1, 2 or 3 heteroatoms independently selected from N, O and S as members of the ring;
R³⁰ represents a hydrogen atom, a linear or branched C₁-C₆ aliphatic radical which may be substituted with 1, 2 or 3 substituents independently selected from F, Cl, Br, —OH, —SH, —O—CH₃, —O—C₂H₅, —NO₂, —CN and —S—CH₃;
R³¹ to R⁴³ represent, independently of each other, a hydrogen atom; a linear or branched, saturated or unsaturated C₁-C₅ aliphatic radical, which may be substituted by 1, 2 or 3 substituents independently selected from F, Cl, Br, —OH, —NH₂, —SH, —O—CH₃, —O—C₂H₅, —NO₂, —CN, —NH—CH₃ and —S—CH₃; a saturated or unsaturated cycloaliphatic radical with 3 to 8 members, which may be substituted by 1, 2 or 3 substituents independently selected from C₁₋₅alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅alkyl, F, Cl, Br, I, —CN, —CF₃, —OCF₃, —SCF₃, —OH, —SH, —NH₂, —NH(C₁₋₅-alkyl), —N(C₁₋₅alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₄-alkyl, —S(═O)₂-phenyl, cyclopropyl, cyclobutyl, cyclopentyl, cyclohexyl, phenyl, phenoxy benzyloxy and benzyl and which optionally may include 1, 2 or 3 heteroatoms independently selected from N, O and S as members of the ring and which may be bonded through a linear or branched C₁-C₆ alkylene group; or an aryl or heteroaryl radical with 5 to 14 members that may be substituted by 1, 2 or 3 substiuents independently selected from —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₄-alkyl), —N(C₁₋₅-alkyl)₂, —NH—C(═O)—C₁₋₅-alkyl, —N(C₁₋₅ alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅alkyl, —S(═O)₂-phenyl, cyclopropyl, cyclobutyl, cyclopentyl, cyclohexyl, phenyl, phenoxy, benzyloxy and benzyl and which may be bonded through a linear or branched C₁-C₆ alkylene, C₂-C₆ alkenylene or C₂-C₆ alkynylene group, and where the heteroaryl radical contains 1, 2 or 3 heteroatoms independently selected from N, O and S as members of the ring;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

Compounds according to formula (II) are known from WO07/054,257 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (II) is/are selected from:
[1] (2-methyl-6-nitro-3H-inden-1-yl)acetic acid
[2] [2-methyl-6-(naphthalene-2-sulphonylamine)-3H-inden-1-yl]acetic acid
[3] [3(Z)-benzylidene-2-methyl-6-(naphthalene-2-sulphonylamine)-3H-inden-1-yl]acetic acid
[4] [2-methyl-4-(naphthalene-2-sulphonylamine)-3H-inden-1-yl]acetic acid
[5] [6-(naphthalene-2-sulphonylamine)-3H-inden-1-yl]acetic acid
[6] [6(5-chloro-3-methylbenzo[b]thiophene-2-sulphonylamine]-2-methyl-3H-inden-1-yl]acetic acid
[7] [2-methyl-6-(naphthalen-1-ylsulfamoyl)-3H-inden-1-yl]acetic acid
[8] N,N-Dimethyl-2-(2-methyl-6-nitro-3H-inden-1-yl)acetamide
[9] 2-(2-Methyl-6-nitro-31+inden-1-yl)-1-pyrrolidin-1-ylethanone
[10] 2-[3(Z)-Benzylidene-2-methyl-6-(naphthalene-2-sulphonylamine)-3H-inden-1-yl]-N,N-dimethylacetamide
[11] N,N-Dimethyl-2-[2-methyl-6-(naphthalene-2-sulphonylamine)-3H-inden-1-yl]acetamide
[12] N-[2-Methyl-3-(2-oxo-2-pyrrolidin-1-ylethyl)-1H-inden-5-yl]naphthalene-2-sulfonamide
[13] N[2-Methyl-1-(2-oxo-2-pyrrolidin-1-ylethyl)-3H-inden-4-yl]naphthalene-2-sulfonamide
[14] N-[3-(2-Oxo-2-pyrrolidin-1-ylethyl)-1H-inden-5-yl]naphthalene-2-sulfonamide
[15] N-[2-Methyl-3-(2-oxo-2-pyrrolidin-1-ylethyl)-1H-inden-5-yl]-5-chloro-3-methyl benzo[b]thiophene-2-sulfonamide
[16] N,N-Dimethyl-2-[2-methyl-6-(naphthalen-1-ylsulfamoyl)-3H-inden-1-yl]acetamide
[17] Dimethyl-[2-(2-methyl-6-nitro-3H-inden-1-yl)ethyl]amine
[18] 3-(2-Dimethylaminoethyl)-2-methyl-1H-inden-5-ylamine
[19] N[3-(2-Dimethylaminoethyl)-2-methyl-1H-inden-5-yl]-6-chloroimidazo[2,1-b]thiazole-5-sulfonamide
[20] N[3-(2-Dimethylaminoethyl)-2-methyl-1H-inden-5-yl]-5-chloro-3-methylbenzo[b]thiophene-2-sulfonamide
[21] N-4-[3-(2-Dimethylaminoethyl)-2-methyl-1H-inden-5-ylsulfamoyl]phenylacetamide
[22] N[3-(2-Dimethylaminoethyl)-2-methyl-1H-inden-5-yl]benzo[1,2,5]thiadiazole-4-sulfonamide
[23] N-Ethyl-N[3-(2-dimethylaminoethyl)-2-methyl-1H-inden-5-yl]-5-chloro-3-methylbenzo[b]thiophene-2-sulfonamide
[24] 4-Amino-N[3-(2-dimethylaminoethyl)-2-methyl-1H-inden-5-yl]benzene sulfonamide
[25] N-[3-(2-Pyrrolidin-1-ylethyl)-2-methyl-1H-inden-5-yl]-2-(4-benzyloxyphenyl)acetamide
[26] 2-Methyl-3-(2-pyrrolidin-1-ylethyl)-1H-inden-5-ylamine
[27] (2-(6-[(5-chloro-3-methylbenzo[b]thiophene-2-sulfonyl)ethylamino]-2-methyl-3H inden-1-yl)ethyl)ethyldimethylammonium iodide
[28] 1-[2-(2-Methyl-6-nitro-3H-inden-1-yl)ethyl]pyrrolidine
[29] N[3-(2-Pyrrolidin-1-ylethyl)-2-methyl-1H-inden-5-yl]-6-chloroimidazo[2,1-b]thiazole-5-sulfonamide
[30] N{4-[3-(2-Pyrrolidin-1-ylethyl)-2-methyl-1H-inden-5-ylsulfamoyl]phenyl}acetamide
[31] N-[3-(2-Pyrrolidin-1-ylethyl)-2-methyl-1H-inden-5-yl]-benzo[1,2,5]thiadiazole-4-sulfonamide
[32] 4-Amino-N-[3-(2-pyrrolidin-1-ylethyl)-2-methyl-1H-inden-5-yl]benzenosulfonamide
[33] N[1 (2)-Benzylidene-3-(2-dimethylaminoethyl)-2-methyl-1H-inden-5-yl]naphthalene-2-sulfonamide
[34] N[3-(2-Dimethylaminoethyl)-2-methyl-1H-inden-5-yl]naphthalene-2-sulfonamide
[35] N[2-Methyl-3-(2-pyrrolidin-1-ylethyl)-1H-inden-5-yl]naphthalene-2-sulfonamide
[36] N[2-Methyl-1-(2-pyrrolidin-1-ylethyl)-3H-inden-4-yl]naphthalene-2-sulfonamide
[37] N[3-(2-Pyrrolidin-1-ylethyl)-1H-inden-5-yl]naphthalene-2-sulfonamide
[38] N[2-Methyl-3-(2-pyrrolidin-1-ylethyl)-1H-inden-5-yl]-5-chloro-3-methylbenzo[b]thiophene-2-sulfonamide
[39] N(Naphthalen-1-yl)-3-(2-dimethylaminoethyl)-2-methyl-1H-indeno-5-sulfonamide
[40] N[3-(2-Hydroxyethyl)-2-methyl-1-inden-5-yl]naphthalene-2-sulfonamide
[41] 6-Chloro-N-{3-[2-(dimethylamino)ethyl]-1,1-dimethyl-1H-inden-5-yl}imidazo[2,1-b] [1,3]thiazole-5-sulfonamide
[42] 5-Chloro-N{3-[2-(dimethylamino)ethyl]-1,1-dimethyl-1H-inden-5-yl}-3-methylbenzo[b]thiophene-2-sulfonamide
[43] N-{3-[2-(Dimethylamino)ethyl]-2-methyl-1H-inden-5-yl}naphthalene-1-sulfonamide
[44] N-{3-[2-(bimethylamino)ethyl]-2-methyl-1H-inden-5-yl}-1-benzothiophene-3-sulfonamide
[45] 6-Chloro-N-[2-methyl-3-(1-methylpyrrolidin-3-yl)-H-inden-5-yl]imidazo[2,1-b] [1,3]thiazole-5-sulfonamide
[46] 6-Chloro-V[2-methyl-3-(1-methylpiperidin-3-yl)-1H-inden-5-yl]imidazo[2,1-b] [1,3]thiazole-5-sulfonamide
[48] 6-Chloro-N-{3-[2-(dimethylamino)ethyl]-1H-inden-5-yl}imidazo[2,1-b] [1,3]thiazole-5-sulfonamide
[49] 6-Chloro-N[3-(2-piperidin-1-ylethyl)-1H-inden-5-yl]imidazo[2,1-b] [1,3]thiazole-5-sulfonamide
[50] 6-Chloro-M[3-(1-methylpyrrolidin-3-yl)-1H-inden-5-yl]imidazo[2,1-b] [1,3] thiazole-5-sulfonamide;
   optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

In another preferred embodiment of the combination of active substances according to the invention, at least one compound (A) or at least one compound (B) or at least one compound (A) and one compound (B) binding to the 5HT6-receptor is/are selected from compounds according to formula (III)

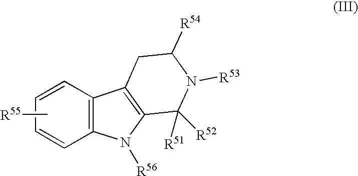

wherein
R⁵¹ and R⁵², identical or different, represent hydrogen, C₁-C₆ alkyl, C₂-C₆ alkenyl, C₂-C₆ alkinyl, aryl, heteroaryl, C₃-C₆ cycloalkyl or C₃-C₆ heterocycloalkyl, optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, independent from one another, each represent a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene, alkenylene or alkinylene group,
   or R⁵¹ and R⁵² together form a spiro substituent of 3-6 carbons;
   R⁵³ represents hydrogen, C₁-C₆ alkyl, C₂₋₆ alkenyl, C₂₋₆ alkinyl, C₃-C₆ cycloalkyl, C₃-C₆ heterocycloalkyl, aryl or heteroaryl; optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR—C(═O)—R^{g}; —NH—C(═O)—O—R′; —NR^{f}—C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
   R⁵⁴ represents hydrogen, CO—NR^{a}R^{b}, CO—OR^{a}, wherein
R^{a} and R^{b}, identical or different, represent hydrogen, C₁-C₆ alkyl, aryl, heteroaryl, C₃-C₆ cycloalkyl, or C₃-C₆ heterocycloalkyl, optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —NR^{f})—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR—C(═O)—R^{g}; —NH—C(═O)—O—R^{f}; —NR^{f}—C(═O)—O—R^{g}; —S(═O)₂—O—R^{f}; an halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
   R⁵⁵ represents NRCSO₂ R^{d}, wherein
R^{c} represents hydrogen or C₁₋₄ alkyl optionally substituted with one or more substituents independently selected from C₁-C₆ alkyl, aryl, cyano, C₁-C₆ alkoxy and trifluoromethyl;

R^{d} represents aryl or heteroaryl optionally substituted with one or more substituents independently selected from —NO₂; —NH₂; —SH; —H; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
   R⁵⁶ represents hydrogen, C₁₋₄ alkyl, aryl, heteroaryl or SO₂R^{e}, wherein
R^{e} represents aryl, heteroaryl, C₃-C₆ cycloalkyl, C₃-C₆ heterocycloalkyl; optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R)—S(═O)₂—R^{g}; —NH-R^{f}; —NR^{f}R^{g}; —C(═O)—NHR^{f}, —C(═O)—NR-R^{g}; S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; an halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
   wherein R^{f} and R^{g}, have the meaning defined above
      optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.
Compounds according to formula (III) are known from WO07/028,460 A1 and are well suitable to be selected for the combination of active substances according to the invention and its respective components of COMPOUND (A) or COMPOUND (B), and thus the content of this publication referred to is in its entirety forming part of the description of this invention by reference.

It is a further preferred embodiment of the combination of active substances according to the invention if the compound/s binding to the 5HT6-receptor according to formula (III) is/are selected from:
[1] 6-chloro-imidazo[2,1-b]thiazole-5-sulfonic acid (2-methyl-2,3,4,9-tetrahydro-1H-β-carbolin-6-yl)-amide;
[2] Benzo[b]thiophene-3-sulfonic acid (2-methyl-2,3,4,9-tetrahydro-1H-β-carbolin-6-yl)-amide;
[3] Naphthalene-1-sulfonic acid (2-methyl-2,3,4,9-tetrahydro-1H-β-carbolin-6-yl)-amide;
[4] 5-Chloro-naphthalene-2-sulfonic acid (2-methyl-2,3,4,9-tetrahydro-1H-β-carbolin-6-yl)-amide;
[5] 5-Chloro-3-methyl-benzo[b]thiophene-2-sulfonic acid (2-methyl-2,3,4,9-tetrahydro-1H-β-carbolin-6-yl)-amide;
[6] Benzo[1,2,5]thiadiazole-4-sulfonic acid (2-methyl-2,3,4,9-tetrahydro-1H-β-carbolin-6-yl)-amide;
[7] N-[4-(2-Methyl-2,3,4,9-tetrahydro-1H-β-carbolin-6-ylsulfamoyl)-phenyl]-acetamide;
[8] 4-Amino-N-(2-methyl-2,3,4,9-tetrahydro-1H-β-carbolin-6-yl)-benzenesulfonamide;
[9] N[4-Methyl-5-(2-methyl-2,3,4,9-tetrahydro-1H-β-carbolin-6-ylsulfamoyl)-thiazol-2-yl]-acetamide;
[10] 5-Dimethylamino-naphthalene-1-sulfonic acid (2-methyl-2,3,4,9-tetrahydro-1H-β-carbolin-6-yl)-amide;
[11] Benzofuran-2-sulfonic acid (2-methyl-2,3,4,9-tetrahydro-1H-β-carbolin-6-yl)-amide;
[12] Naphthalene-2-sulfonic acid (2-methyl-2,3,4,9-tetrahydro-1H-βcarbolin-6-yl)-amide;
   optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

Further compounds binding to the 5HT6 receptor suitable for the combination of active substances according to the invention and its respective components of COMPOUND (A) or COMPOUND (B) are also known from WO06/069807 A1 and WO06/069808 and thus the content of this publication referred to is in its entirety forming part of the description of this invention by reference.

The combination of active substances according to the invention and its respective components of COMPOUND (A) or COMPOUND (B) are physiologically active compounds with a suitable therapeutic window. They are non-toxic. Accordingly the combination of active substances according to the invention may be used in pharmaceutical formulations and used in the treatment of various diseases exemplified below.

Thus, a further aspect of the invention is a medicament comprising a combination of active substances according to the invention and optionally at least one pharmaceutical adjuvant.

Another further aspect of the invention thus is also a pharmaceutical formulation comprising a combination of active substances according to the invention and optionally at least one pharmaceutical adjuvant. This pharmaceutical formulation may also be formulated into a medicament.

In a preferred embodiment of the medicament according to the invention the medicament is for the treatment of Peripheral Nervous System Disorders, or Central Nervous System Disorders, especially Central Nervous System Disorders.

In another preferred embodiment of the medicament according to the invention the medicament is for the treatment of cognitive disorders, memory disorders, senile dementia processes, such as Alzheimer's, Parkinson's and/or Huntington's Disease, attention deficit disorder, such as infantile hyperkinesia (ADHD, attention deficit/hyperactivity disorder), epilepsy, anxiety, panic, depression, psychosis, pain, schizophrenia; or for the improvement/enhancement of cognition.

In a preferred embodiment of the Medicament according to the invention the medicament is for the treatment of cognitive disorders, degenerative disorders, memory disorders, ADHD (attention deficit/hyperactivity disorder), Alzheimer's disease, senile dementia process, learning disabilities caused by degenerative disorders, learning disabilities caused by non-degenerative disorders, memory or cognitive dysfunction such as mild cognitive impairment, age-related cognitive decline, cerebral senility, vascular dementia, AIDS-associated dementia, electric shock induced amnesia, memory impairment associated with depression or anxiety, cognitive defects in Parkinson's disease, Down's syndrome, stroke, traumatic brain injury, Huntington's disease, and attention deficit disorder, infantile hyperkinesia (ADHD, attention deficit/hyperactivity disorder); especially ADHD, or for the improvement/enhancement of cognition.

Thus, a further aspect of the invention is the use of a combination of active substances according to the invention for the manufacture of a medicament for the treatment of Peripheral Nervous System Disorders, or Central Nervous System Disorders, especially Central Nervous System Disorders.

“Treatment” as used in this application is defined as the treatment of a disease or of a medically relevant symptom, but also includes the prevention of the symptom or disease preventive/prophylactic activity during or before the development of the symptom or disease.

In a preferred embodiment of the use according to invention the medicament manufactured is for the treatment of cognitive disorders, memory disorders, senile dementia processes, such as Alzheimers, Parkinson's and/or Huntington's Disease, attention deficit disorder, such as infantile hyperkinesia (ADHD, attention deficit/hyperactivity disorder), epilepsy, anxiety, panic, depression, psychosis, pain, schizophrenia; or for the improvement/enhancement of cognition.

In another preferred embodiment of the use according to invention the medicament manufactured is for the treatment of cognitive disorders, degenerative disorders, memory disorders, ADHD (attention deficit/hyperactivity disorder), Alzheimer's disease, senile dementia process, learning disabilities caused by degenerative disorders, learning disabilities caused by non-degenerative disorders, memory or cognitive dysfunction such as mild cognitive impairment, age-related cognitive decline, cerebral senility, vascular dementia, AIDS-associated dementia, electric shock induced amnesia, memory impairment associated with depression or anxiety, cognitive defects in Parkinson's disease, Down's syndrome, stroke, traumatic brain injury, Huntington's disease, and attention deficit disorder, infantile hyperkinesia (ADHD, attention deficit/hyperactivity disorder); especially ADHD, or for the improvement/enhancement of cognition.

The medicament may be in any form suitable for the application to humans and/or animals, preferably mammals, and can be produced by standard procedures known to those skilled in the art. The composition of the medicament may vary depending on the route of administration.

The medicament of the present invention may e.g. be administered parentally in combination with conventional injectable liquid carriers, such as water or suitable alcohols. Conventional pharmaceutical adjuvants for injection, such as stabilizing agents, solubilizing agents, and buffers, may be included in such injectable compositions. These medicaments may preferably be injected intramuscularly, intraperitoneally, or intravenously.

Medicaments according to the present invention may also be formulated into orally administrable compositions containing one or more physiologically compatible pharmaceutical adjuvants like carriers or excipients, in solid or liquid form. These compositions may contain conventional ingredients such as binding agents, fillers, lubricants, and acceptable wetting agents. The compositions may take any convenient form, such as tablets, pellets, capsules, lozenges, aqueous or oily solutions, suspensions, emulsions, or dry powdered form suitable for reconstitution with water or other suitable liquid medium before use, for immediate or controlled release.

The liquid oral forms for administration may also contain certain other pharmaceutical adjuvants like additives such as sweeteners, flavoring, preservatives, and emulsifying agents. Non-aqueous liquid compositions for oral administration may also be formulated, containing e.g. edible oils. Such liquid compositions may be conveniently encapsulated in e.g., gelatin capsules in a unit dosage amount.

The compositions (or medicaments) of the present invention may also be administered topically or via a suppository.

The above mentioned compositions (or medicaments) include preferably 1 to 60% by weight of the combination of active substances according to the invention, and 40 to 99% by weight of the appropriate pharmaceutical vehicle(s).

The daily dosage applied to a patient/mammal of the combination of active substances according to the invention and also of each of its respective Compounds (A) or (B) being 5HT6 ligands may vary depending on factors that have their basis in the respective species or other factors, such as age, weight or degree of illness and so forth. The daily dosage for mammals including humans of the combination of active substances according to the invention and also of each of its respective Compounds (A) or (B) usually ranges from 1 milligram to 2000 milligram, preferably 1 to 1500 mg, more preferably 1 to 1000 mg of substance to be administered during one or several intakes.

As a further aspect the invention also provides a method of treatment for cognitive disorders, memory disorders or degenerative brain disorders by applying to a mammal or patient in need thereof a suitable amount of a 5HT6 ligand acting as a partial or full agonist and of a 5HT6 ligand acting as a full antagonist or inverse agonist either separately or in the form of a combination of active substances according to the invention. Thus, either the 5HT6 ligand acting as a partial or full agonist is applied after the 5HT6 ligand acting as a full antagonist or inverse agonist has been applied or the 5HT6 ligand acting as a partial or full agonist is applied before the 5HT6 ligand acting as a full antagonist or inverse agonist has been applied. Or, in a further embodiment the 5HT6 ligand acting as a partial or full agonist is applied at the same time (or approximately the same time) when and the 5HT6 ligand acting as a full antagonist or inverse agonist is applied, whereas this may be achieved through the same pharmaceutical pathway or even the same medicament (as a combination of the active substances according to the invention).

The following figures and examples are provided to illustrate the claimed invention and are not meant in any way to limit it.

### FIGURES

FIG. 1: Effect of co-administration of COMPOUND 1, a 5HT6 ligand acting as agonist on the 5HT6 receptor and the 5HT6 ligand SB-271046 acting as antagonist on the 5HT6 receptor. As can be clearly seen the combined treatmen of COMPOUND 1 and SB-271046 did result in a quite pronounced effect, even an synergistic effect, over the use of each of the substances alone, which at these doses were either not or very low effective in other dose response studies (see below). *p<0.05 and **p<0.01 Student's Paired t-Test from the novel object within same treatment.

FIG. 2: Dose response trials using either A) COMPOUND 1 (at 1.25, 2.5, 5 or 10 mg/kg i.p.) or B) SB-271046 (at 5 or 10 mg/kg i.p.) the maximum effect was achieved with COMPOUND 1 at 5 mg/kg and for SB-271046 at 10 mg/kg, with neither COMPOUND 1 at 1 mg/kg nor SB-271046 at 5 mg/kg being significantly effective. *p<0.05 and **p<0.01 Student's Paired t-Test from the novel object within same treatment.

### EXAMPLES

For exemplifying the of the combination SB-271046 was used. SB-271046 is a well-known compound binding to the 5HT6 receptor and acting as an antagonist.

The other compound used was 6-Chloro-imidazo[2,1-b]thiazole-5-sulfonic acid [3-(2-dimethylamino-ethyl)-1H-indol-5-yl]-amide (hereinafter called COMPOUND 1):

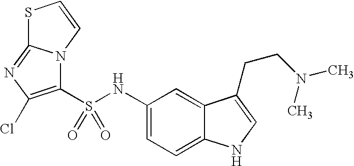

This compound is known from WO 03/42175 A1 and is an agonist with a very low Kᵢ in binding to the 5HT6 receptor.

### Example 1

### Novel Object Discrimination Trial

### Methods

Adult male Lister Hooded rats (Charles River, UK) weighing 200-350 g at the start of the experiment were housed in groups of four on a 12:12 h light:dark cycle (lights on at 07:00 h). Food and water were available ad libitum throughout the study, and the room temperature (21±2° C.) and relative humidity (45-65%) were kept constant. A group of rats (n=12 each) received injection of a sub-effective dose of COMPOUND 1 (1 mg/kg i.p.) or vehicle (0.5% methylcellulose in saline, 2 ml/kg), either alone or combined with SB-271046 (5 mg/kg). Each drug combination was administered to all rats in the group over a period of 4 weeks in a random order using a seven day behavioural test interval.

The two trial novel object discrimination paradigm utilised, was as described by Ennaceur and Delacour, (1988) with minor modification (King et al., 2004b; Woolley et al., 2003). The twelve open field test arenas used for object discrimination were clear perspex boxes (39×23.5 cm with 24.5 cm high walls) to which each rat was habituated for 60 minutes the day prior to test days. On the test day the first drug was administered −40 minutes and the second drug −20 minutes before the familiarisation trial. Therefore, 20 minutes after the injection each rat received 3 minutes acclimatisation to the perspex box in absence of objects which was then followed by the 3 minute familiarisation trial and a second 3 minute choice trial following a 4 hour inter-trial interval. During both trials, exploration of each object was defined as the time spent (s) sniffing (within 1 cm of it with active vibrissae), licking, chewing or touching the object with the nose.

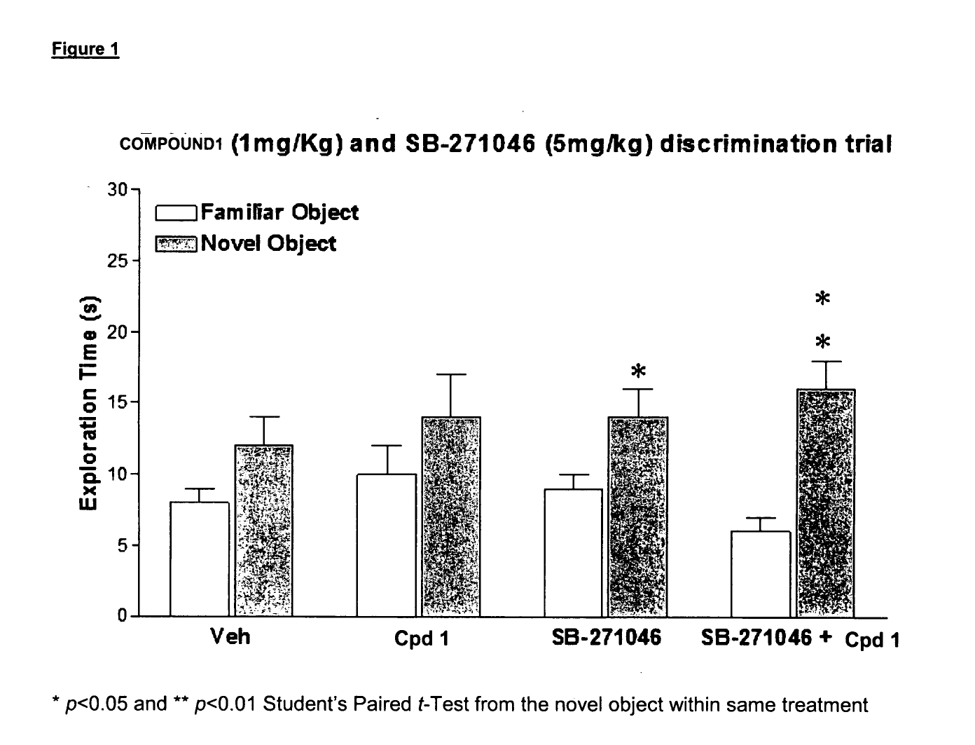

As can be clearly seen in FIG. 1 the combined treatmen of COMPOUND 1 and SB-271046 did result in a quite pronounced effect, even an synergistic effect, over the use of each of the substances alone.

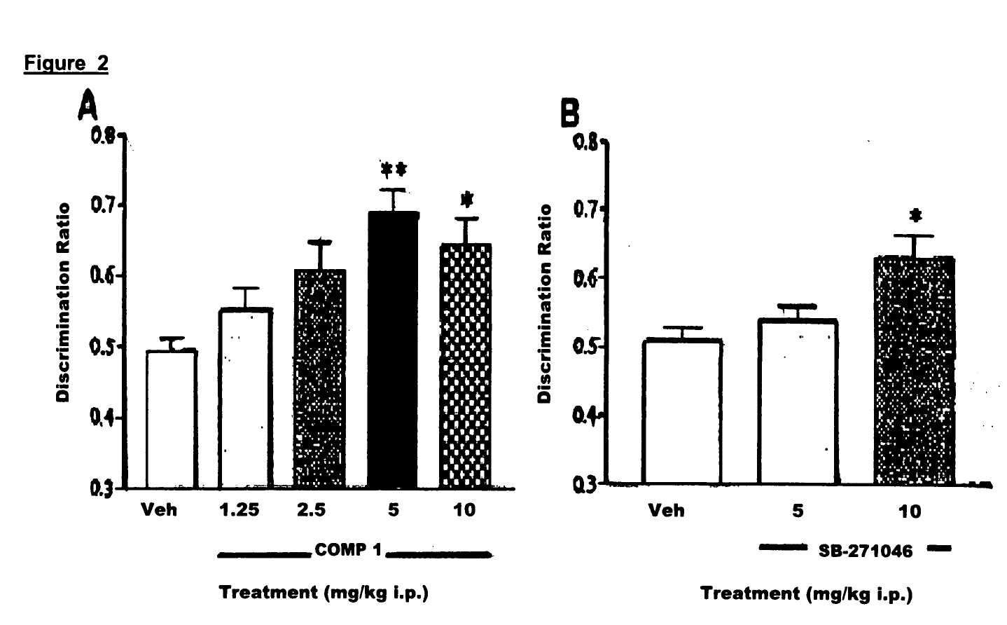

In previous dose response trials according to above description (on the test day the respective drug was administered −20 minutes before the familiarisation trial) using either COMPOUND 1 (at 1.25, 2.5, 5 or 10 mg/kg i.p.) or SB-271046 (at 5 or 10 mg/kg i.p.) the maximum effect was achieved with COMPOUND 1 at 5 mg/kg and for SB-271046 at 10 mg/kg, with neither COMPOUND 1 at 1 mg/kg nor SB-271046 at 5 mg/kg being significantly effective (See FIGS. 2 A) and B)).

1. A combination of active substances comprising
at least one compound (A)
being selected from compounds binding to the 5HT6-receptor and acting as an full agonist or partial agonist.
and at least one compound (B)
being selected from compounds binding to the 5HT6-receptor and acting as an antagonist or inverse agonist;

2. A combination of active substances according to claim 1 wherein at least one compound (A) or at least one compound (B) or at least one compound (A) and one compound (B) are on the 5HT6 receptor having an Kᵢ value of ≦5000 nM, preferably of ≦1000 nM, more preferably of ≦500 nM.

3. A combination of active substances according to claim 1 wherein at least one compound (A) or at least one compound (B) or at least one compound (A) and one compound (B) are on the 5HT6 receptor having an Kᵢ value of ≦500 nM, preferably of ≦250 nM, more preferably of ≦100 nM, most preferably of ≦50 nM.

4. A combination of active substances according to claim 1 wherein at least one compound (A) or at least one compound (B) or at least one compound (A) and one compound (B) are binding to the 5HT6 receptor with a higher affinity than to the 5-HT1A or 5HT7 receptor; preferably binding with an affinity higher by a factor of at least 10, preferably with an affinity higher by a factor of at least 30, more preferably with an affinity higher by a factor of at least 50, most preferably with an affinity higher by a factor of at least 100.

5. A combination of active substances according to claim 1 wherein at least one compound (A) or at least one compound (B) or at least one compound. (A) and one compound (B) are binding to the 5HT6 receptor with a higher affinity than to the 5-HT1A or 5HT7 receptor; preferably binding with an affinity higher by a factor of at least 10, preferably with an affinity higher by a factor of at least 30, more preferably with an affinity higher by a factor of at least 50, most preferably with an affinity higher by a factor of at least 100.
and
wherein at least one compound (A) or at least one compound (B) or at least on compound (A) and one compound (B) are binding to the 5HT6 receptor with a Kᵢ value of ≦5000 nM, preferably of ≦1000 nM, more preferably of ≦500 nM, or of ≦250 nM, or of ≦100 nM, or most preferably of ≦50 nM.

6. A combination of active substances according to any of claims 2 to 5 wherein at least one compound (A) and at least one compound (B) are on the 5HT6 receptor having the Kᵢ value and/or have the affinity according to any of claims 2 to 5.

7. A combination of active substances according to any of claims 2 to 5, wherein at least one compound (A) or at least one compound (B) or at least one compound (A) and one compound (B) binding to the 5HT6-receptor is/are selected from SB-271046, Ro 04-6790, SB-357134, SB-399885, SB-399885T, 5-methoxy-2-phenyl-N,N-dimethyl triptamine (BGC20-761), or 2-ethyl-5-methoxy-N,N-dimethyltriptamine, WAY-181187, WAY-466;
preferably wherein at least one compound (A) is selected from 2-ethyl-5-methoxy-N,N-dimethyltriptamine, WAY-181187, WAY-466 and/or at least one compound (B) is selected from SB-271046, SB-271046-A Ro 04-6790, SB-357134, SB-399885, SB-399885T, 5-methoxy-2-phenyl-N,N-dimethyl triptamine (BGC20-761).

8. A combination of active substances according to any of claims 2 to 7, wherein at least one compound (A) or at least one compound (B) or at least one compound (A) and one compound (B) binding to the 5HT6-receptor is/are selected from sulfonamide compounds according to formula (I), 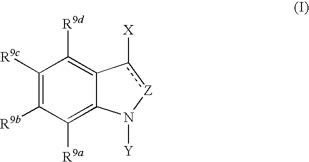
wherein
=Z either represents ═C(R¹⁰) or —CH₂ or ═N;
and wherein
either
Y represents —S(O₂)R¹³, while X represents R¹;
or
X represents R¹, while Y represents (CH₂), —R¹¹ or
Y represents R¹, while X represents (CH₂)ₙ—R¹²;
while
one of R^{9a}, R^{9b}, R^{9c}, or R^{9d} represents N(R³S(O₂)-A, or N—S(O₂)-A)₂;
and wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—C₁₋₅-alkyl, C(═O)—O—C₁₋₅-alkyl, —O—C(═O)—C₁₋₅-alkyl, oxo (═O), F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy, benzyl, and pyridinyl and which may be condensed with a saturated or unsaturated mono- or bicyclic ring system, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and whereby the rings of the ring system are 5-6- or 7-membered and may contain 1, 2 or 3 heteroatom(s) independently selected from the group consisting of nitrogen, oxygen and sulfur and which may be bonded via a linear or branched C₁₋₄ alkylene, C₂₋₆ alkenylene or C₂₋₆ alkinylene group which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃ and wherein the heteroaryl radical contains 1, 2 or 3 heteroatom(s) independently selected from the group consisting of nitrogen, oxygen and sulfur as ring member(s);
R¹ represents hydrogen, a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or an optionally at least monosubstituted alkyl-aryl radical;
R³ represents a hydrogen atom; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d}— if not N(R³)—S(O₂)-A or N-(S(O₂)-A)₂— independently from one another, each represent a hydrogen atom; —NO₂; —NH₂; —SH; —OH; —CN; —C(═O)H; —C(═O)—R; —C(═O)—O—R′; —OR′; —SR′; —N(R′)-S(═O)₂—R″; —NH—R′; —NR⁺R⁺⁺; F; Cl, Br; I; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or a 5- to 14-membered aryl or heteroaryl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may be bonded via a linear or branched C₁₋₆ alkylene group and wherein the heteroaryl radical contains 1, 2 or 3 heteroatom(s) independently selected from the group consisting of nitrogen, oxygen and sulfur as ring member(s);
R¹⁰ represents a hydrogen atom; —NO₂; —NH₂; —SH; —OH; —CN; —C(═O)—OH; —O—R′; —S—R′; —C(═O)—OR″; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═OYOH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and whereby the rings of the ring system are 5-, 6- or 7-membered and may contain 1, 2 or 3 heteroatom(s) as ring member(s) independently selected from the group consisting of nitrogen, oxygen and sulfur;
R¹² represents R¹¹,
or
represents 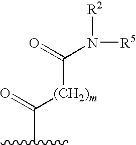
with n being 0;
or represents —C(OC₁₋₄₅-alkyl)-(CH₂)ₘ—R¹¹;
R¹³ represents a saturated or unsaturated, optionally at least mono-substituted cycloaliphatic radical, or —CHR′R″;
n being 0, 1, 2, 3 or 4;
m being 0, 1, 2, 3 or 4;
R′ and R″ identical or different, each represents a saturated or unsaturated, linear or branched, optionally at least mono-substituted aliphatic radical;
R* and R* identical or different, each represents a saturated or unsaturated, linear or branched, optionally at least mono-substituted aliphatic radical;
or
R* and R** together with the connecting nitrogen form an optionally at least monosubstituted heterocyclyl radical
R² represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
or
R² and R⁵ together with the bridging nitrogen form a saturated, unsaturated or aromatic 3- to 9-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)OH, —C(═O)—C₁₋₅-alkyl; —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and whereby the rings of the ring system are 5-6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

9. A combination of active substances according to claim 8, wherein at least one compound (A) or at least one compound (B) or at least one compound (A) and one compound (B) binding to the 5HT6-receptor is/are selected from sulfonamide compound according to formula (Ia), 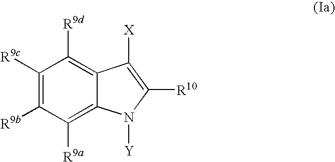
wherein
either
Y represents S(O₂)—R³, while X represents R′;
or
X represents R¹, while Y represents (CH₂)ₙ—R¹¹ or
Y represents R¹, while X represents (CH₂), —R¹²;
while
one of R^{9a}, R^{9b}, R^{9c}, or R^{9d} represents N(R³)—S(O₂)-A,
and wherein
A, R¹, R³, R^{9a}, R^{9b}, R^{9c}, R^{9d}, R¹⁰, R¹¹, R¹², R¹³ and n are as defined in claim 2.

10. A combination of active substances according to any of claims 8 or 9, wherein at least one compound (A) or at least one compound (B) or at least one compound (A) and one compound (B) binding to the 5HT6-receptor is/are selected from sulfonamide compounds according to formula (I), or (Ia)
wherein
=Z either represents ═C(R¹⁰) or —CH₂ or ═N;
and wherein
either
Y represents —S(O₂)—R¹³, while X represents R¹;
or
X represents R¹, while Y represents (CH₂)ₙ—R¹¹ or
Y represents R¹, while X represents (CH₂)ₙ—R¹²;
while
one of R^{9a}, R^{9b}, R^{9c}, or R^{9d} represents N(R³)—S(O₂)-A, or N—(S(O₂)-A)₂;
and wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d}—if not N(R³)—S(O₂)-A or N—(S(O₂)-A)₂— independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₄ alkyl radical which may be substituted with 1, 2 or 3 substituent(s) independently selected from the group consisting of F, Cl, Br, —OH, —NH₂, and —SH;
R¹⁰ represents a hydrogen atom; a linear or branched optionally at least mono-substituted C₁₋₅-alkyl radical;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
R¹² represents R¹¹,
or
represents 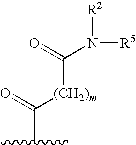
with n being 0;
or represents —C(OC₁₋₅-alkyl)-(CH₂)ₘ, —R¹;
R¹³ represents a saturated or unsaturated, optionally at least mono-substituted C₅₋₇-cycloaliphatic radical, or —CHR′R″; with R′ and R″ identical or different, each representing a saturated or unsaturated, linear or branched, optionally at least mono-substituted C₁₋₅-alkyl radical;
n being 0, 1, 2, 3 or 4;
m being 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur.

11. A combination of active substances according to any of claims 8 to 10, wherein at least one compound (A) or at least one compound (B) or at least one compound (A) and one compound (B) binding to the 5HT6-receptor is/are selected from sulfonamide compounds according to formula (Ib), or (Ic), 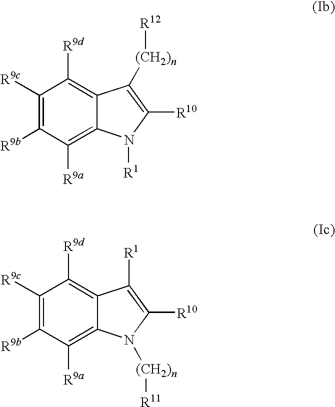
and wherein R¹, R^{9a}, R^{9b}, R^{9c}, R^{9d}, R¹⁰, R¹¹, R¹² and n have the meaning given in claims 2 or 4.

12. A combination of active substances according to claim 11, wherein at least one compound (A) or at least one compound (B) or at least one compound (A) and one compound (B) binding to the 5HT6-receptor selected from sulfonamide compounds according to formula (Ib) is a compound according to formulas (Iba), (Ibb) or (Ibc), 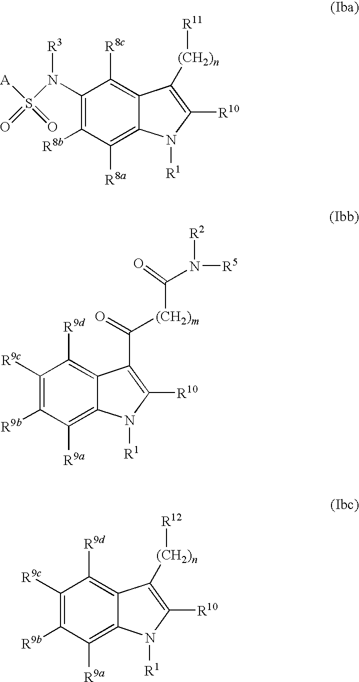
and wherein A, R¹, R², R³, R⁵, R^{9a}, R^{9b}, R^{9c}, R^{9d}, R¹⁰, R¹¹, R², m and n have the meaning given in claims 2 or 4 and
wherein
R^{8a}, R^{8b}, R^{8c} independently from one another, each represent a hydrogen atom; —NO₂; —NH₂; —SH; —OH; —CN; —C(═O)—H; —C(═O)—R′; —C(═O)—O—R′; —OR′; —SR′; —N(R′)-S(═O)₂—R″; —NH—R′; —NR*R**; F; Cl, Br; I; a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or a 5- to 14-membered aryl or heteroaryl radical, which may be substituted with 1, 2 or 3 substituent(s) independently selected from the group consisting of —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)C₁₋₅-alkyl, —N(C₁₋₅-alkyl)-C(═O)—C₁₋₅-alkyl, —NO₂, —CHO, —CF₂H, —CFH₂, —C(═O)—NH₂, —C(═O)-NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy and benzyl and which may be bonded via a linear or branched C₁₋₆ alkylene group and wherein the heteroaryl radical contains 1, 2 or 3 heteroatom(s) independently selected from the group consisting of nitrogen, oxygen and sulfur as ring member(s);
preferably R^{8a}, R^{8b}, R^{8c} independently from one another, each represent a hydrogen atom; —NO₂; —NH₂; —SH; —OH; —CN; —C(═O)—H; —C(═O)O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH.

13. A combination of active substances according to claim 11, wherein at least one compound (A) or at least one compound (B) or at least one compound (A) and one compound (B) binding to the 5HT6-receptor selected from sulfonamide compounds according to formula (Ic) is a compound according to formulas (Ica), (Icb), (Icc), or (Icd), 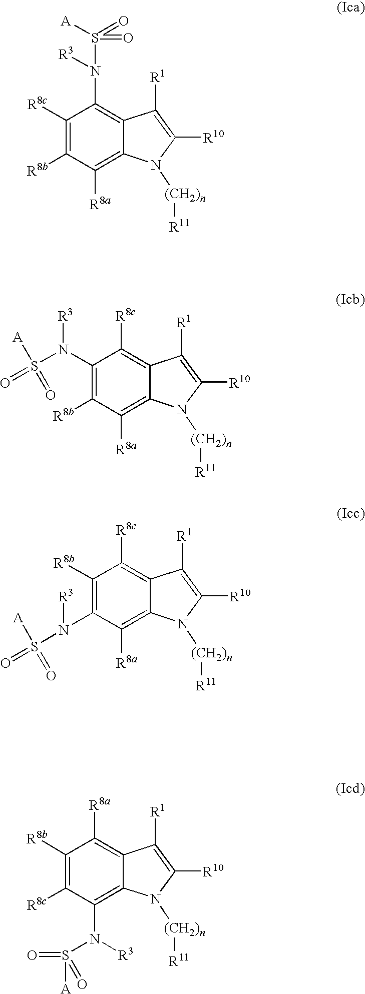
and wherein A, R¹, R³, R¹⁰, R¹¹, R¹², m and n have the meaning given in claims 2 or 4 and wherein R^{8a}, R^{8b}, R^{8c} have the meaning given in claim 6.

14. A combination of active substances according to claim 12, wherein at least one compound (A) or at least one compound (B) or at least one compound (A) and one compound (B) binding to the 5HT6-receptor is/are selected from sulfonamide compounds according to formula (Iba) 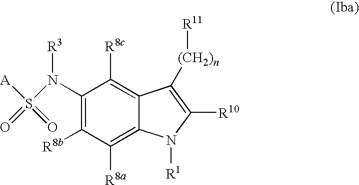
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{8a}, R^{8b}, R^{8c} each represent a hydrogen atom;
R¹⁰ represents a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

15. A combination of active substances according to claim 14, wherein the compound/s binding to the 5HT6-receptor is/are selected from the following group of sulfonamide compounds according to formula (Iba) consisting of:
[1] N-[3-(2-diethylaminoethyl)-1H-indol-5-yl]-5-chloro-3-methylbenzo[b]thiophene-2-sulphonamide.
[2] N-[3-(2-diethylaminoethyl)-1H-indol-5-yl]naphthalene-1-sulphonamide.
[3] Hydrochloride N-[3-(2-diethylaminoethyl)-1H-indol-5-yl]naphthalene-1-sulphonamide.
[4] N-[3-(2-diethylaminoethyl)-1H-indol-5-yl]-3,5-dichlorobenzenesulphon amide.
[5] N-[3-(2-diethylaminoethyl)-1H-indol-5-yl]-4-phenylbenzenesulphonamide.
[6] N-[3-(2-diethylaminoethyl)-1H-indol-5-yl]-5-chlorothiophene-2-sulphonamide.
[7] N-[3-(2-dimethylaminoethyl)-1H-indol-5-yl]-5-chloro-3-methylbenzo[b]thiophene-2-sulphonamide.
[8] N-[3-(2-dimethylaminoethyl)-1H-indol-5-yl]naphthalene-1-sulphonamide.
[9] N-[3-(2-dimethylamino-ethyl)-1H-indol-5-yl]-6-chloroimidazo[2,1-b]thiazol-5-sulphonamide.
[10] N-[3-(1-methylpiperidin-4-yl)-1H-indol-5-yl]-5-chloro-3-methylbenzo[b]thiophene-2-sulphonamide.
[11] N-[3-(1-methylpiperidin-4-yl)-1H-indol-5-yl]-5-chloro-3-methylbenzo[b]thiophene-2-sulphonamide hydrochloride.
[12] N-[3-(1-methylpiperidin-4-yl)-1H-indol-5-yl]naphthalene-1-sulphonamide.
[13] N-[3-(1-methylpiperidin-4-yl)-1H-indol-5-yl]naphthalene-1-sulphonamide hydrochloride.
[14] N-[3-(1-methylpiperidin-4-yl)-1H-indol-5-yl]-5-chlorothiophene-2-sulphonamide.
[15] N-[3-(1-methylpiperidin-4-yl)-1H-indol-5-yl]-4-phenylbenzenesulphonamide.
[16] N-[3-(1-methylpiperidin-4-yl)-1H-indol-5-yl]quinoline-8-sulphonamide.
[17] N-[3-(2-diethylaminoethyl)-1H-indol-5-yl]naphthalene-2-sulphonamide.
[18] N-[3-(1-methyl-1,2,3,6-tetrahydropyridin-4-yl)-1H-indol-5-yl]naphthalene 1-sulphonamide.
[19] N-[3-(4-methylpiperazin-1-yl)methyl-1H-indol-5-yl]-5-chloro-3-methylbenzo[b]thiophene-2-sulphonamide.
[20] N-[3-(2-dimethylaminoethyl)-1H-indol-5-yl]-5-(2-pyridil)thiophene-2-sulphonamide.
[21] N-[3-(2-dimethylaminoethyl)-1H-indol-5-yl]-2,1,3-benzothiadiazol-4-sulphonamide.
[22] N-[3-(2-dimethylaminoethyl)-1H-indol-5-yl]quinoline-8-sulphonamide.
[23] N-[3-(2-dimethylaminoethyl)-1H-indol-5-yl]-5-chloronaphthalene-2-sulphonamide.
[24] N-[3-(2-dimethylaminoethyl)-1H-indol-5-yl]-4-phenoxybenzenesulphonamide.
[25] N-[3-(2-dimethylaminoethyl)-1H-indol-5-yl]-4-phenylbenzenesulphonamide.
[26] N-[3-(2-diethylaminoethyl)-1H-indol-5-yl]-N-ethyl-naphthalene-2-sulphonamide.
[27] N-{3-[2-(morpholin-4-yl)ethyl]-1H-indol-5-yl}-5-chloro-3-methylbenzo[b]thiophene-2-sulphonamide.
[28] N-{3-[2-(morpholin-4-yl)ethyl]-1H-indol-5-yl}naphthalene-1-sulphonamide.
[29] N-[3-(2-diethylaminoethyl)-1H-indol-5-yl]naphthalene-2-sulphonamide.
[30] N-[3-dimethylaminomethyl-1H-indol-5-yl]-5-chloro-3-methylbenzo[b]thiophene-2-sulphonamide.
[31] N-[3-(2-dipropylaminoethyl)-1H-indol-5-yl]naphthalene-1-sulphonamide.
[32] N-[3-(2-dipropylaminoethyl)-1H-indol-5-yl]-5-chloro-3-methylbenzo[b]thiophene-2-sulphonamide.
[33] N-[3-(2-dibutylaminoethyl)-1H-indol-5-yl]-5-chloro-3-methylbenzo[b]thiophene-2-sulphonamide.
[34] N-[3-(2-dibutylaminoethyl)-1H-indol-5-yl]naphthalene-1-sulphonamide.
[35] N-[3-(2-diethylaminoethyl)-1H-indol-5-yl]-5-chloronaphthalene-1-sulphonamide.
[36] N-[3-(2-diethylaminoethyl)-1H-indol-5-yl]-trans-β-styrenesulphonamide.
[37] N-[3-(4-methylpiperazin-1-yl)methyl-1H-indol-5-yl]-trans-β-styrenesulphonamide.
[38] N-[3-(octahydroindolizin-7-yl)-1H-indol-5-yl]-5-chloro-3-methylbenzo[b]thiophene-2-sulphonamide.
[39] N-[3-(2-diethylaminoethyl)-1H-indol-5-yl]-6-chloroimidazo[2,1-b]thiazol-5-sulphonamide.
[40] N-{3-[2-(morpholin-4-yl)ethyl]-1H-indol-5-yl}naphthalene-2-sulphonamide.
[41] N-[3-(4-methylpiperazin-1-yl)methyl-1H-indol-5-yl]-α-toluenesulphonamide.
[42] N-[3-(3-diethylaminopropyl)-1H-indol-5-yl]naphthalene-2-sulphonamide.
[43] N-[3-(3-diethylaminopropyl)-1H-indol-5-yl]-5-chloro-3-methylbenzo[b]thiophene-2-sulphonamide.
[44] N-{3-[2-(pyrrolidin-1-yl)ethyl]-1H-indol-5-yl}-5-chloro-3-methylbenzo[b]thiophene-2-sulphonamide.
[45] N-{3-[2-(pyrrolidin-1-yl)ethyl]-1H-indol-5-yl}naphthalene-1-sulphonamide.
[46] N-{3-[2-(pyrrolidin-1-yl)ethyl]-1H-indol-5-yl}naphthalene-2-sulphonamide.
[47] N-[3-(2-dipropylaminoethyl)-1H-indol-5-yl]naphthalene-2-sulphonamide.
[48] N-[3-(2-dimethylaminoethyl)-1H-indol-5-yl]-5-chloronaphthalene-1-sulphonamide.
[49] N-[3-(2-dimethylaminoethyl)-1H-indol-5-yl]naphthalene-2-sulphonamide.
[50] N-{3-[2-(morpholin-4-yl)ethyl]-1H-indol-5-yl}quinoline-8-sulphonamide.
[51] N-{3-[2-(morpholin-4-yl)ethyl]-1H-indol-5-yl}-4-phenylbenzenesulphonamide.
[52] N-[3-(4-methylpiperazin-1-yl)ethyl-1H-indol-5-yl]naphthalene-2-sulphonamide.
[53] N-[3-(4-methylpiperazin-1-yl)ethyl-1H-indol-5-yl]-5-chloronaphthalene-1-sulphonamide;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

16. A combination of active substances according to claim 12, wherein at least one compound (A) or at least one compound (B) or at least one compound (A) and one compound (B) binding to the 5HT6-receptor is/are selected from sulfonamide compounds according to formula (Ibb) 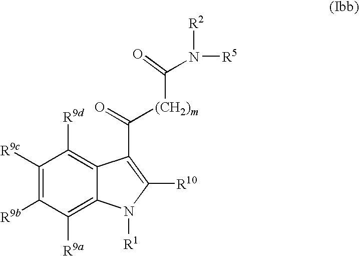
wherein
one of R^{9a}, R^{9b}, R^{9c}, or R^{9d} represents N(R³)—S(O₂)-A, or N—(S(O₂)-A)₂;
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃;
R^{9a}, R^{9b}, R^{9c}, R^{9d}— if not N(R³—S(O₂)-A or N—(S(O₂)-A)₂— independently from one another, each represent a hydrogen atom; —NO₂; —NH₂; —SH; —OH; —CN; —C(═O)H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH;
R¹⁰ represents a hydrogen atom; a linear or branched optionally at least mono-substituted C₁₋₅-alkyl radical;
m represents 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁-r-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

17. A combination of active substances according to claim 16, wherein the compound/s binding to the 5HT6-receptor is/are selected from the following group of sulfonamide compounds according to formula (Ibb) consisting of:
2-[5-(5-Chloro-3-methyl-benzo[b]thiophene-2-sulfonylamino)-1H-indol-3-yl]-N,N-diethyl-2-oxoacetamide.
N,N-Diethyl-2-[5-naphthalene-2-sulfonylamino)-1H-indol-3-yl]-2-oxo-acetamide.
N,N-Diethyl-2-[5-(naphtalene-1-sulfonylamino)-1H-indol-3-yl]-2-oxo-acetamide.
2-[5-Biphenyl-4-sulfonylamino)-1H-indol-3-yl]-N,N-diethyl-2-oxo-acetamide.
N,N-Diethyl-2-oxo-2-[5-(quinoline-8-sulfonylamino)-1H-indol-3-yl]-acetamide.
N,N-Dimethyl-2-[5-(naphthalene-2-sulfonylamino)-1H-indol-3-yl]-2-oxo-acetamide.
N,N-Dimethyl-2-[5-(naphtalene-1-sulfonylamino)1H-indol-3-yl]-2-oxo-acetamide.
2-[5-(5-Chloro-3-methyl-benzo[b]thiophene-2-sulfonylamino)-1H-indol-3-yl]-N,N-dimethyl-2-oxo-acetamide.
2-[5-(6-Chloro-imidazo[2,1-b]thiazole-5-sulfonylamino)-1H-indol-3-yl]-N,N-diethyl-2-oxo-acetamide.
2-[5-(6-Chloro-imidazo[2,1-b]thiazole-5-sulfonylamino)-1H-indol-3-yl]-N,N-dimethyl-2-oxo-acetamide.
N,N-Dimethyl-2-[4-(naphthalene-1-sulfonylamino)-1H-indol-3-yl]-2-oxo-acetamide.
2-[4-(5-Chloro-3-methyl-benzo[b]thiophene-2-sulfonylamino)-1H-indol-3-yl]-N,N-dimethyl-2-oxo-acetamide.
2-[4-(6-Chloro-imidazo[2,1-b]thiazole-5-sulfonylamino)-1H-indol-3-yl]-N,N-dimethyl-2-oxo-acetamide.
N,N-Dimethyl-2-[5-[(4-fluoro-3-methyl-phenyl)-1-sulfonylamino]-1H-indol-3-yl]-2-oxo-acetamide.
5-(3-Dimethylaminooxalyl-1H-indol-5-ylsulfamoyl)-3-methyl-benzofuran-2-carboxylic acid ethyl ester.
2-[5-(Biphenyl-4-sulfonylamino)-1H-indol-3-yl]-N,N-dimethyl-2-oxo-acetamide.
N,N-Dimethyl-2-oxo-2-[5-(2-oxo-2,3-dihydro-benzoxazole-6-sulfonylamino)-1H-indol-3-yl]-acetamide.
N,N-Dimethyl-2-oxo-2-[5-(2-oxo-2,3-dihydrobenzo[d]thiazole-6-sulfonamido)-1H-indol-3-yl]acetamide.
2-[5-[(4-Cyclohexyl-phenyl)-1-sulfonylamino]-1H-indol-3-yl]-N,N-dimethyl-2-oxo-acetamide.
N,N-Dimethyl-2-[5-[(4-phenoxy-phenyl)-1-sulfonylamino]-1H-indol-3-yl]-2-oxo-acetamide.
2-(5-(5-chloro-3-methylbenzo[b]thiophene-2-sulfonamidoy2-methyl-1H-indol-3-yl)-N,N-dimethyl-2-oxoacetamide.
2-(5-(6-chloroimidazo[2,1-b]thiazole-5-sulfonamido)-2-methyl-1H-indol-3-yl)-N,N-dimethyl-2-oxoacetamide.
2-(6-(5-chloro-3-methylbenzo[b]thiophene-2-sulfonamido-1H-indol-3-yl)-N,N-dimethyl-2-oxoacetamide.
N,N-dimethyl-2-(6-naphthalene-3-sulfonamido)-1H-indol-3-yl)-2-oxoacetamide.
2-(6-(biphenyl-4-sulfonamido)-1H-indol-3-yl)-N,N-dimethyl-2-oxoacetamide.
N,N-dimethyl-2-(6-(naphthalene-1-sulfonamido)-1H-indol-3-yl)-2-oxoacetamide.
N,N-dimethyl-2-(6-(2-naphthalen-1-yl)ethylsulfonamido)-1H-indol-3-yl)-2-oxoacetamide.
N,N-dimethyl-2-oxo-2-(6-4-phenoxyphenylsulfonamido)-1H-indol-3-yl)acetamide.
2-(6-(3,4-dichlorothiophene-2-sulfonamido)-1H-indol-3-yl)-N,N-dimethyl-2-oxoacetamide.
2-(6-(3,5-dichlorophenylsulfonamido)-1H-indol-3-yl)-N,N-dimethyl-2-oxoacetamide.
2-(6-(1-chloronaphthalene-6-sulfonamido)-1H-indol-3-yl)-N,N-dimethyl-2-oxoacetamide.
2-(6-(6-chloroimidazo[2,1-b]thiazole-5-sulfonamido)-1H-indol-3-yl)-N,N-dimethyl-2-oxoacetamide.
N,N-diethyl-2-(2-methyl-5-(5-methyl-1-phenyl-1H-pyrazole-4-sulfonamido)-1H-indol-3-yl)-2-oxoacetamide.
N,N-diethyl-2-(2-methyl-5-(1,3,5-trimethyl-1H-pyrazole-4-sulfonamido)-1H-indol-3-yl)-2-oxoacetamide;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

18. A combination of active substances according to claim 12, wherein at least one compound (A) or at least one compound (B) or at least one compound (A) and one compound (B) binding to the 5HT6-receptor is/are selected from sulfonamide compounds according to formula (Ibc) 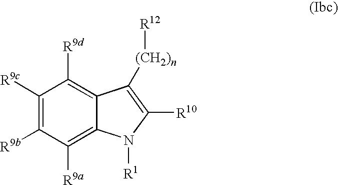
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents hydrogen;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³— if present—represents hydrogen;
R^{9a}, R^{9b}, R^{9c}, R^{9d}—if not N(R³)S(O₂)-A or N—(S(O₂)-A)₂— independently from one another, each represent a hydrogen atom; —NO₂; —NH₂; —SH; —OH; —CN; —C(═O)H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₄₅ alkyl radical which may be substituted with 1, 2 or 3 substituent(s) independently selected from the group consisting of F, Cl, Br, —OH, —NH₂, and —SH;
with the proviso that one of R^{9a}, R^{9b}, R^{9c}, or R^{9d} represents N(R³)—S(O₂)-A, or N—(S(O₂)-A)₂;
R¹⁰ represents a hydrogen atom; a linear or branched optionally at least mono-substituted C₁₋₅-alkyl radical; preferably R¹⁰ represents hydrogen;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
R¹² represents R¹¹, or represents —C(OC₁₋₅-alkyl)-(CH₂)ₘ—R¹¹;
n being 0, 1, 2, 3 or 4;
m being 0, 1, 2, or 3;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

19. A combination of active substances according to claim 18, wherein the compound/s binding to the 5HT6-receptor is/are selected from the following group of sulfonamide compounds according to formula (Ibc) consisting of:
5-chloro-N-(3-(2-diethylamino)ethyl)-1H-indol-6-yl)-3-methylbenzo[b]thiophene-2-sulfonamide
N-(3-(2-diethylamino)ethyl)-1H-indol-6-yl)naphthalene-2-sulfonamide
N-(3-(2-diethylamino)ethyl)-1H-indol-6-yl)naphthalene-1-sulfonamide
6-chloro-N-(3-(2-diethylamino)ethyl)-1H-indol-6-yl)imidazo[2,1-b]thiazole-5-sulfonamide
N-(3-2-diethylamino)ethyl)-1H-indol-6-yl)-4-phenylbenzenesulfonamide
N-(3-2-diethylamino)ethyl)-1H-indol-6-yl)-4-phenoxybenzenesulfonamide
3,5-dichloro-N-(3-(2-(diethylamino)ethyl)-1H-indol-6-yl)benzenesulfonamide
4,5-dichloro-N-(3-(2-(diethylamino)ethyl)-1H-indol-6-yl)thiophene-2-sulfonamide
5-chloro-N-(3-(2-diethylamino)ethyl)-1H-indol-6-yl)naphthalene-1-sulfonamide
5-chloro-N-(3-(2-dimethylamino)ethyl)-1H-indol-6-yl)-3-methylbenzo[b]thiophene-2-sulfonamide
N-(3-(2-(dimethylamino)ethyl)-1H-indol-6-yl)naphthalene-2-sulfonamide
N-(3-(2-(dimethylamino)ethyl)-1H-indol-6-yl)naphthalene-1-sulfonamide
6-chloro-N-(3-(2-dimethylamino)ethyl)-1H-indol-6-yl)imidazo[2,1-b]thiazole-5-sulfonamide
N-(3-2-dimethylamino)ethyl)-1H-indol-6-yl)-4-phenylbenzenesulfonamide
N-(3-(2-dimethylamino)ethyl)-1H-indol-6-yl)-2-(naphthalen-1-yl)ethanesulfonamide
N-(3-2-dimethylamino)ethyl)-1H-indol-6-yl)-4-phenoxybenzenesulfonamide
3,5-dichloro-N-(3-(2-(dimethylamino)ethyl)-1H-indol-6-yl)benzenesulfonamide
4,5-dichloro-N-(3-(2-(dimethylamino)ethyl)-1H-indol-6-yl)thiophene-2-sulfonamide
5-chloro-N-(3-(2-dimethylamino)ethyl)-1H-indol-6-yl)naphthalene-1-sulfonamideo
5-chloro-N-(3-(2-dimethylamino)-1-ethoxyethyl)-1H-indol-7-yl)-3-methylbenzo[b]thiophene-2-sulfonamide
5-chloro-N-(3-(2-dimethylamino)ethyl)-1H-indol-7-yl)-3-methylbenzo[b]thiophene-2-sulfonamide
7-bis(5-chloro-3-methylbenzo[b]thiophene-2-sulfonyl)amino-3-(2-(diethylamino)-1-ethoxyethyl)-1H-indole
5-chloro-N-(3-(2-(diethylamino)-1-ethoxyethyl)-1H-indol-7-yl)-3-methylbenzo[b]thiophene-2-sulfonamide
7-bis(5-chloro-3-methylbenzo[b]thiophene-2-sulfonyl)amino-3-(2-(dimethylamino)ethyl)-1H-indole
7-bis(5-chloro-3-methylbenzo[b]thiophene-2-sulfonyl)amino-3-(2-(diethylamino)ethyl)-1H-indole
5-chloro-N-(3-(2-(diethylamino)ethyl)-1H-indol-7-yl)-3-methylbenzo[b]thiophene-2-sulfonamide
7-bis(6-chloroimidazo[2,1-b]thiazol-5-ylsulfonyl)amino-3-2-dimethylamino)ethyl)-1H-indole
N-(3-2-(dimethylamino)ethyl)-1H-indol-4-yl)-4-biphenylsulfonamide
N-(3-(2-(dimethylamino)ethyl)-1H-indol-4-yl)-4-phenoxybenzenesulfonamide
3,5-dichloro-N-(3-(2-(dimethylamino)ethyl)-1H-indol-4-yl)benzenesulfonamide
5-chloro-N-(3-(2-(dimethylamino)ethyl)-1H-indol-4-yl)-3-methylbenzo[b]thiophene-2-sulfonamide
N-(3-(2-(dimethylamino)ethyl)-1H-indol-4-yl)naphthalene-1-sulfonamide
5-chloro-N-(3-(2-(dimethylamino)ethyl)-1H-indol-4-yl)naphthalene-2-sulfonamide
N-(3-(2-(dimethylamino)ethyl)-1H-indol-4-yl)naphthalene-2-sulfonamide
6-chloro-N-(3-(2-dimethylamino)ethyl)-1H-indol-4-yl)imidazo[2,1-b]thiazole-5-sulfonamide
N-(3-(2-(dimethylamino)ethyl)-1H-indol-4-yl)-2-(naphthalen-1-yl)ethanesulfonamide
6-bis(6-chloroimidazo[2,1-b]thiazol-5-ylsulfonyl)amino-3-2-(dimethylamino)ethyl)-1H-indole
6-bis(3,5-dichlorobenzenesulfonyl)amino-3-(2-(dimethylamino)ethyl)-H-indole
6-bis(4,5-dichlorothiophene-2-sulfonyl)amino-3-(2-(dimethylamino)ethyl)-1H-indole
6-bis(5-chloro-3-methylbenzo[b]thiophene-2-sulfonyl)amino-3-(2-(dimethylamino)-1-ethoxyethyl)-1H-indole
N-(3-(2-diethylamino)ethyl)-7-methoxy-1H-indol-5-yl)naphthalene-2-sulfonamide
N-(3-(2-diethylamino)ethyl)-7-methoxy-1H-indol-5-yl)benzo[c][1,2,5]thiadiazole-4-sulfonamide
6-chloro-N-(3-(2-diethylamino)ethyl)-7-methoxy-1H-indol-5-yl)imidazo[2,1-b]thiazole-5-sulfonamide
ethyl 6-(5-chloro-3-methylbenzo[b]thiophene-2-sulfonamido)-3-(1-methylpiperidin-4-yl)-1H-indole-5-carboxylate
N-(5-bromo-3-(2-(pyrrolidin-1-yl)ethyl)-1H-indol-7-yl)-6-chloroimidazo[2,1-b]thiazole-5-sulfonamide
N-(4-bromo-3-(1-methylpiperidin-4-yl)-1H-indol-6-yl)naphthalene-1-sulfonamide
N-(7-bromo-3-(2-dimethylamino)ethyl)-1H-indol-5-yl)benzofuran-2-sulfonamide
N-(7-methoxy-3-(2-(pyrrolidin-1-yl)ethyl)-1H-indol-5-yl)benzo[c][1,2,5]thiadiazole-4-sulfonamide
N-(7-methoxy-3-(2-(pyrrolidin-1-yl)ethyl)-1H-indol-5-yl)naphthalene-2-sulfonamide
6-chloro-N-(7-methoxy-3-(2-(pyrrolidin-1-yl)ethyl)-1H-indol-5-yl)imidazo[2,1-b]thiazole-5-sulfonamide;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively;
preferably is selected from:
N-(3-(2-(diethylamino)ethyl)-7-methoxy-1H-indol-5-yl)naphthalene-2-sulfonamide,
N-(3-(2-(diethylamino)ethyl)-7-methoxy-1H-indol-5-yl)benzo[c][1,2,5]thiadiazole-4-sulfonamide,
6-chloro-N-(3-(2-(diethylamino)ethyl)-7-methoxy-1H-indol-5-yl)imidazo[2,1-b]thiazole-5-sulfonamide,
ethyl 6-(5-chloro-3-methylbenzo[b]thiophene-2-sulfonamido)-3-(1-methylpiperidin-4-yl)-1H-indole-5-carboxylate,
N-(5-bromo-3-2-pyrrolidin-1-yl)ethyl)-1H-indol-7-yl)-6-chloroimidazo[2,1-b]thiazole-5-sulfonamide,
N-(4-bromo-3-(1-methylpiperidin-4-yl)-1H-indol-6-yl)naphthalene-1-sulfonamide,
N-(7-bromo-3-(2-(dimethylamino)ethyl)-1H-indol-5-yl)benzofuran-2-sulfonamide,
N-(7-methoxy-3-(2-(pyrrolidin-1-yl)ethyl)-1H-indol-5-yl)benzo[c][1,2,5]thiadiazole-4-sulfonamide,
N-(7-methoxy-3-(2-(pyrrolidin-1-yl)ethyl)-1H-indol-5-yl)naphthalene-2-sulfonamide and
6-chloro-N-(7-methoxy-3-(2-(pyrrolidin-1-yl)ethyl)-1H-indol-5-yl)imidazo[2,1-b]thiazole-5-sulfonamide.

20. A combination of active substances according to claim 13, wherein at least one compound (A) or at least one compound (B) or at least one compound (A) and one compound (B) binding to the 5HT6-receptor is/are selected from sulfonamide compounds according to formula (Ica) 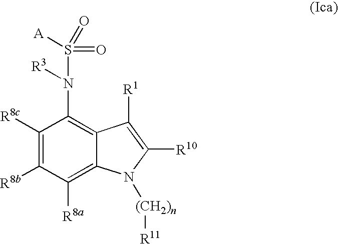
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
R¹⁰ represents a hydrogen atom; a linear or branched optionally at least mono-substituted C₁₋₅-alkyl radical; preferably R¹⁰ represents a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4; preferably n being 2;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

21. A combination of active substances according to claim 20, wherein the compound/s binding to the 5HT6-receptor is/are selected from the following group of sulfonamide compounds according to formula (Ica) consisting of:
[1] N-[1-(2-dimethylaminoethyl)-1H-indole-4-yl]-5-chloro-3-methylbenzo[b]thiophene-2-sulfonamide,
[2] N-[1-(2-dimethylaminoethyl)-1H-indole-4-yl]-naphtalene-2-sulfonamide,
[3] N-[1-(2-dimethylaminoethyl)-1H-indole-4-yl]-naphtalene-1-sulfonamide,
[4] N-[1-(2-dimethylaminoethyl)-1H-indole-4-yl]-4-phenylbenzenesulfonamide,
[5] N-[1-(2-dimethylaminoethyl)-1H-indole-4-yl]-2-(naphtalene-1-yl)-ethanesulfonamide,
[6] N-[1-(2-dimethylaminoethyl)-1H-indole-4-yl]-4-phenoxybenzenesu Hfonamide,
[7] N-[1-(2-dimethylaminoethyl)-1H-indole-4-yl]-3,5-dichlorobenzenesulfonamide and
[8] 6-chloro-N-[1-(2-dimethylaminoethyl)-1H-indol-4-yl]-imidazo[2,1-b]thiazole-5-sulfonamide
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

22. A combination of active substances according to claim 13, wherein at least one compound (A) or at least one compound (B) or at least one compound (A) and one compound (B) binding to the 5HT6-receptor is/are selected from sulfonamide compounds according to formula (Icb) 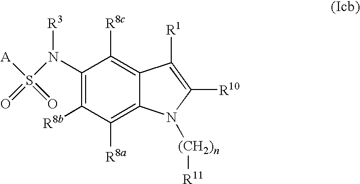
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
R¹⁰ represents a hydrogen atom; a linear or branched optionally at least mono-substituted C₁₋₅-alkyl radical; preferably R¹⁰ represents a hydrogen atom or methyl;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4; preferably n being 2;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

23. A combination of active substances according to claim 22, wherein the compound/s binding to the 5HT6-receptor is/are selected from the following group of sulfonamide compounds according to formula (Icb) consisting of:
[1] N-[1-(2-dimethylaminoethyl)-1H-indole-5-yl]-5-chloro-3-methylbenzo[b]thiophene-2-sulfonamide,
[2] N-[1-(2-dimethylaminoethyl)-1H-indole-5-yl]-naphthalene-2-sulfonamide,
[3] N-[1-(2-dimethylaminoethyl)-1H-indole-5-yl]-naphthalene-1-sulfonamide,
[4] N-[1-(2-dimethylaminoethyl)-1H-indole-5-yl]-5-chloronaphthalene-1-sulfonamide,
[5] N-[1-(2-dimethylaminoethyl)-1H-indole-5-yl]-benzenesulfonamide,
[6] N-[1-(2-dimethylaminoethyl)-1H-indole-5-yl]-quinoline-8-sulfonamide,
[7] N-[1-(2-dimethylaminoethyl)-1H-indole-5-yl]-4-phenoxybenzenesulfonamide,
[8] N-[1-(2-dimethylaminoethyl)-1H-indole-5-yl]-4-methylbenzenesulfonamide,
[9] N-[1-(2-dimethylaminoethyl)-1H-indole-5-yl]-5-chlorothiophene-2-sulfonamide,
[10] N-[1-(2-dimethylaminoethyl)-1H-indole-5-yl]-benzo[1,2,5]thiadiazole-4-sulfonamide,
[11] N-[1-(2-dimethylaminoethyl)-1H-indole-5-yl]-6-chloroimidazo[2,1-b]thiazole-5-sulfonamide,
[12] N-[1-(2-dimethylaminoethyl)-1H-indole-5-yl]-3,5-dichlorobenzenesulfonamide,
[13] N-[1-(2-dimethylaminoethyl)-1H-indole-5-yl]-3-bromobenzenesulfonamide,
[14] N-[1-(2-dimethylaminoethyl)-1H-indole-5-yl]-3-nitrobenzenesulfonamide,
[15] N-[1-(2-dimethylaminoethyl)-1H-indole-5-yl]-1-phenylmethanesulfonamide,
[16] N-[1-(2-pyrrolidine-1-yl-ethyl)-1H-indole-5-yl]-naphthalene-2-sulfonamide,
[17] N-[1-(2-pyrrolidine-1-yl-ethyl)-1H-indole-5-yl]-naphthalene-1-sulfonamide,
[18] N-[1-(2-pyrrolidine-1-yl-ethyl)-1H-indole-5-yl]-5-chloro-3-methylbenzo[b]thiophene-2-sulfonamide,
[19] trans-N-[1-(2-dimethylaminoethyl)-1H-indole-5-yl]-2-phenylethenesulfonamide,
[20] N-[1-(2-dimethylaminoethyl)-1H-indole-5-yl]-4,5-dichlorothiophene-2-sulfonamide,
[21] N-[1-(2-dimethylaminoethyl)-1H-indole-5-yl]-4-acetylbenzenesulfonamide,
[22] N-[1-(2-dimethylaminoethyl)-1H-indole-5-yl]-4-bromobenzenesulfonamide,
[23] N-[1-2-dimethylaminoethyl)-1H-indole-5-yl]-4-methoxybenzenesulfonamide,
[24] N-[3-2-diethylaminoethyl)-1H-indole-5-yl]-5-chloro-3-methylbenzo[b]thiophene-2-sulfonamide,
[25] N-[1-(2-dimethylaminoethyl)-1H-indole-5-yl]-4-nitrobenzenesulfonamide,
[26] N-[1-(2-dimethylaminoethyl)-1H-indole-5-yl]-4-fluorobenzenesulfonamide,
[27] N-[1-(2-diethylaminoethyl)-1H-indole-5-yl]-6-chloroimidazo[2,1-b]thiazole-5-sulfonamide,
[28] N-[1-(2-pyrrolidine-1-yl-ethyl)-1H-indole-5-yl]-]-6-chloroimidazo[2,1-b]thiazole-5-sulfonamide,
[29] N-(1-(2-(diethylamino)ethyl)-1H-indol-5-yl)-naphthalene-2-sulfonamide,
[30] N-(1-(2-(diethylamino)ethyl)-1H-indol-5-yl)-naphthalene-1-sulfonamide,
[31] N-(1-(2-(diethylamino)ethyl)-1H-indol-5-yl)-4-phenylbenzenesulfonamide,
[32] 5-chloro-N-(1-(2-(dimethylamino)ethyl)-2-methyl-1H-indol-5-yl)-3-methylbenzo[b]thiophene-2-sulfonamide,
[33] N-(1-(2-(dimethylamino)ethyl)-2-methyl-1H-indol-5-yl)-naphthalene-2-sulfonamide,
[34] N-(1-(2-(dimethylamino)ethyl)-2-methyl-1H-indol-5-yl)-naphthalene-1-sulfonamide,
[35] 6-chloro-N-(1-(2-(dimethylamino)ethyl)-2-methyl-1H-indol-5-yl)imidazo[2,1-b]thiazole-5-sulfonamide,
[36] N-(1-(2-dimethylamino)ethyl)-2-methyl-1H-indol-5-yl)-4-phenylbenzenesulfonamide,
[37] N-(1-(2-dimethylamino)ethyl)-2-methyl-1H-indol-5-yl)-2-(naphth-1-yl)-ethanesulfonamide,
[38] N-(1-(2-dimethylamino)ethyl)-2-methyl-1H-indol-5-yl)-4-phenoxy-benzenesulfonamide,
[39] 3,5-dichloro-N-(1-(2-(dimethylamino)ethyl)-2-methyl-1H-indol-5-yl)-benzenesulfonamide,
[40] N-(1-(2-dimethylamino)ethyl)-2-methyl-1H-indol-5-yl)benzo[b]thiophene-3-sulfonamide,
[41] N-(1-(4-diethylamino)ethyl)-1H-indol-5-yl)benzo[b]thiophene-3-sulfonamide and
[42] N-(1-(2-(dimethylamino)ethyl)-1H-indol-5-yl)benzo[b]thiophene-3-sulfonamide,
[43] 5-chloro-3-methyl-N-(1-(3-(piperidin-1-yl)propyl)-1H-indol-5-yl)benzo[b]thiophene-2-sulfonamide,
[44] N-(1-3-piperidin-1-yl)propyl)-1H-indol-5-yl)naphthalene-2-sulfonamide,
[45] N-(1-(3-piperidin-1-yl)propyl)-1H-indol-5-yl)naphthalene-1-sulfonamide,
[46] 6-chloro-N-(1-(3-piperidin-1-yl)propyl)-1H-indol-5-yl)imidazo[2,1-b]thiazole-5-sulfonamide,
[47] 4-phenyl-N-(1-(3-(piperidin-1-yl)propyl)-1H-indol-5-yl)benzenesulfonamide,
[48] 2-(naphth-1-yl)-N-(1-(3-(piperidin-1-yl)propyl)-1H-indol-5-yl)ethanesulfonamide,
[49] 4-phenoxy-N-(1-(3-(piperidin-1-yl)propyl)-1H-indol-5-yl)benzenesulfonamide,
[50] 3,5-dichloro-N-(1-(3-(piperidin-1-yl)propyl)-1H-indol-5-yl)benzenesulfonylamide,
[51] 4,5-dichloro-N-(1-(3-(piperidin-1-yl)propyl)-1H-indol-5-yl)thiophene-2-sulfonamide and
[52] 5-chloro-N-(1-(3-piperidin-1-yl)propyl)-1H-indol-5-yl)naphthalene-1-sulfonamide,
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

24. A combination of active substances according to claim 13, wherein at least one compound (A) or at least one compound (B) or at least one compound (A) and one compound (B) binding to the 5HT6-receptor is/are selected from sulfonamide compounds according to formula (Icc) 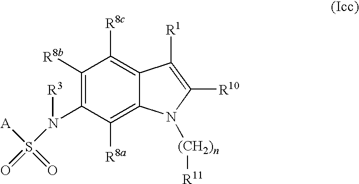
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
R¹⁰ represents a hydrogen atom; a linear or branched optionally at least mono-substituted C₁₋₅-alkyl radical; preferably R¹⁰ represents a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4; preferably n being 2;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁-10 aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

25. A combination of active substances according to claim 24, wherein the compound/s binding to the 5HT6-receptor is/are selected from the following group of sulfonamide compounds according to formula (Icc) consisting of:
[1] N-[1-(2-Dimethylaminoethyl)-1H-indol-6-yl]-5-chloro-3-methylbenzo[b]thiophene-2-sulfonamide,
[2] N-[1-(2-Dimethylaminoethyl)-1H-indol-6-yl]-naphthalene-2-sulfonamide,
[3] N-[1-(2-Dimethylaminoethyl)-1H-indol-6-yl]-naphthalene-1-sulfonamide,
[4] N-[1-(2-Dimethylaminoethyl)-1H-indol-6-yl]-6-chloroimidazo[2,1-b]thiazole-5-sulfonamide,
[5] N-[1-(2-Dimethylaminoethyl)-1H-indol-6-yl]-4-phenylbenzenesulfonamide,
[6] N-[1-(2-Dimethylaminoethyl)-1H-indol-6-yl]-2-(naphthalene-1-yl)-ethanesulfonamide,
[7] N-[1-(2-Dimethylaminoethyl)-1H-indol-6-yl]-4-phenoxybenzenesulfonamide,
[8] N-[1-(2-Dimethylaminoethyl)-1H-indol-6-yl]-3,5-dichlorobenzenesulfonamide,
[9] 5-Chloro-3-methyl-N-[1-[2-(pyrrolidin-1-yl)ethyl-1H-indol-6-yl]-benzo[b]thiophene-2-sulfonamide,
[10] N-(1-[2-(Pyrrolidin-1-yl)ethyl]-1H-indol-6-yl]-napthalene-2-sulfonamide,
[11] N-[1-[2-Pyrrolidin-1-yl]ethyl]-1H-indol-6-yl]-naphthalene-1-sulfonamide,
[12] 6-Chloro-N-[1-[2-(pyrrolidin-1-yl)ethyl]-1H-indol-6-yl]-imidazo[2,1-b]thiazole-5-sulfonamide,
[13] 4-Phenyl-N-(1-(2-(pyrrolidin-1-yl)ethyl)-1H-indol-6-yl)-benzenesulfonamide
[14] 2-(Naphthyl-1-yl)-N-(1-(2-(pyrrolidin-1-yl)ethyl)-1H-indol-6-yl)-ethansulfonamide,
[15] 4-Phenoxy-N-(1-(2-(pyrrolidin-1-yl)ethyl)-1H-indol-6-yl)-benzenesulfonamide and
[16] 3,5-Dichloro-N-(1-(2-pyrrolidin-1-yl)-1H-indol-6-yl)-benzenesulfonamide,
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

26. A combination of active substances according to claim 13, wherein at least one compound (A) or at least one compound (B) or at least one compound (A) and one compound (B) binding to the 5HT6-receptor is/are selected from sulfonamide compounds according to formula (Icd) 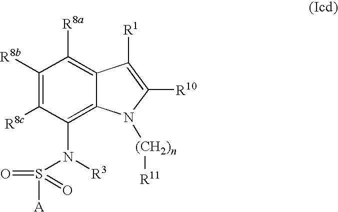
wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
R^{8a}, R^{8b}, R^{8c} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{8a}, R^{8b}, R^{8c} each represent a hydrogen atom;
R¹⁰ represents a hydrogen atom; a linear or branched optionally at least mono-substituted C₁₋₅-alkyl radical; preferably R¹⁰ represents a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4; preferably n being 2;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulphur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

27. A combination of active substances according to claim 26, wherein the compound/s binding to the 5HT6-receptor is/are selected from the following group of sulfonamide compounds according to formula (Icd) consisting of:
[1] N-[1-(2-dimethylaminoethyl)-1H-indole-7-yl]-naphtalene-1-sulfonamide,
[2] N-[1-(2-dimethylaminoethyl)-1H-indole-7-yl]-5-chloro-3-methylbenzo[b]thiophene-2-sulfonamide,
[3] N-[1-(2-dimethylaminoethyl)-1H-indole-7-yl]-4-phenylbenzenesulfonamide and [4] N-[1-(2-dimethylaminoethyl)-1H-indole-7-yl]-6-chloroimidazo[2,1-b]thiazole-5-sulfonamide
[5] 5-chloro-3-methyl-N-(1-(2-(pyrrolidin-1-yl)ethyl)-1H-indol-7-yl)-benzo[b]thiophen-2-sulfonamide,
[6] N-(1-(2-pyrrolidin-1-yl)ethyl)-1H-indol-7-yl)naphthalene-1-sulfonamide,
[7] 6-chloro-N-(1-(2-(pyrroldin-1-yl)ethyl)-1H-indol-7-yl)imidazo[2,1-b]thiazole-5-sulfonamide and
[8] 2-(naphth-1-yl)-N-(1-(2-pyrrolidin-1-yl)ethyl)-1H-indol-7-yl)ethansulfonamide
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

28. A combination of active substances according to any of claims 8 or 9, wherein at least one compound (A) or at least one compound (B) or at least one compound (A) and one compound (B) binding to the 5HT6-receptor is/are selected from sulfonamide compounds according to formula (Id) 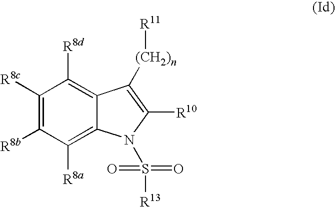
wherein
R^{8a}, R^{8b}, R^{8c}, R^{8d} independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH;
R¹⁰ represents a hydrogen atom; a linear or branched optionally at least mono-substituted C₁₋₅-alkyl radical; preferably R² represents H;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively

29. A combination of active substances according to claim 28, wherein the compound/s binding to the 5HT6-receptor is/are selected from the following group of sulfonamide compounds according to formula (Id) consisting of:
[1] 1-Cyclohexanesulfonyl-3-(1-methyl-1,2,3,6-tetrahydropyridine-4-yl)-5-nitro-1H-indole,
[2] 5-Chloro-1-cyclohexanesulfonyl-3-(1-methyl-1,2,3,6-tetrahydropyridine-4-yl)-1H-indole,
[3] 5-Amino-1-cyclohexanesulfonyl-3-(1-methyl-1,2,3,6-tetrahydropyridine-4-yl)-1H-indole and
[4] 1-Cyclohexanesulfonyl-5-fluoro-3-(1,2,3,5,8,8a-hexahydro-indolizine-7-yl)-1H-indole hydrochloride;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

30. A combination of active substances according to any of claims 8 or 9, wherein at least one compound (A) or at least one compound (B) or at least one compound (A) and one compound (B) binding to the 5HT6-receptor is/are selected from sulfonamide compounds according to formula (Ie) 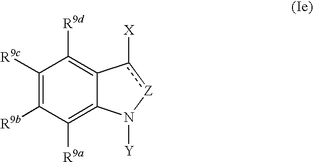
wherein
wherein
=Z either represents —CH₂ or ═N;
and wherein
X represents R¹, while Y represents (CH₂)ₙ—R¹¹ or
Y represents R¹, while X represents (CH₂)ₙ—R¹¹;
while
one of R^{9a}, R^{9b}, R^{9c}, or R^{9d} represents N(R³)—S(O₂)-A;
and wherein
A represents a 5- to 14-membered aryl, alkyl-aryl, heterocyclyl or alkyl-heterocyclyl radical, which may be substituted with 1, 2 or 3 substituent(s) independently selected from the group consisting of —CF₃, C₁₋₅-alkyl, —O—C₁₋₅-alkyl, —C(═O)—OH, —C(═O)—C₁₋₅-alkyl, C(═O)—O—C₁₋₅-alkyl, oxo (═O), F, Cl, Br, I, —CN, —OCF₃, —OH, —SH, —NH₂, —NH(C₁₋₅-alkyl), —N(C₁₋₅-alkyl)₂, —NO₂, —CHO, —CF₂H, —CFH₂, cyclopropyl, cyclobutyl, cyclopentyl, cyclohexyl, phenyl, phenoxy, benzyl, and pyridinyl;
R¹ represents hydrogen, a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; or benzyl; preferably R¹ represents a hydrogen atom;
R³ represents a hydrogen atom; a linear or branchedC₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; preferably R³ represents a hydrogen atom;
R^{9a}, R^{9b}, R^{9c}, R^{9d}— if not N(R³)—S(O₂)-A—independently from one another, each represent a hydrogen atom; —NO₂; —NH₂; —SH; —OH; —CN; —C(═O)—H; —C(═O)—O—C₁₋₅-alkyl; —C(═O)—C₁₋₅-alkyl; —O—C₁₋₅-alkyl; —S—C₁₋₅-alkyl; —F; Cl, Br; I; a linear or branched C₁₋₅ alkyl radical which may be substituted with 1, 2 or 3 substituent(s) independently selected from the group consisting of F, Cl, Br, —OH, —NH₂, and —SH; preferably R^{9a}, R^{9b}, R^{9c}, R^{9d}— if not N(R³)—S(O₂)-A—each represent a hydrogen atom;
R¹¹ represents NR²R⁵ or a saturated or unsaturated 3-, 4-, 5-, 6-, 7- or 8-membered cycloaliphatic radical, which may be substituted with 1, 2 or 3 substituent(s) independently selected from the group consisting of C₁₋₅-alkyl, —O—C₁₋₅-alkyl, F, Cl, Br, I, —CF₃, —OCF₃, —OH, —NH₂, —CF₂H, —CFH₂, cyclopropyl, cyclobutyl, cyclopentyl, cyclohexyl, phenyl, phenoxy and benzyl and which may optionally contain 1, 2 or 3 heteroatom(s) independently selected from the group consisting of nitrogen, oxygen and sulfur as ring member(s) and which may be condensed with a saturated or unsaturated mono- or bicyclic ring system which may be substituted with 1, 2 or 3 substituent(s) independently selected from the group consisting of C₁₋₅-alkyl, —O—C₁₋₅-alkyl, —F, Cl, Br, I, —CN, —CF₃, —OCF₃, —OH, —SH, —NH₂, —CF₂H, —CFH₂, and whereby the rings of the ring system are 5-, 6- or 7-membered and may contain 1, 2 or 3 heteroatom(s) as ring member(s) independently selected from the group consisting of nitrogen, oxygen and sulfur;
n being 0, 1, 2, 3 or 4;
R² represents a hydrogen atom; or a linear or branched C₁₋₅ alkyl radical which may be substituted with 1, 2 or 3 substituent(s) independently selected from the group consisting of F, Cl, Br, —OH, —NH₂, —SH, —O—CH₃, —O—C₂H₅, —NO₂, —CN, —NH—CH₃ and —S—CH₃; optionally at least monosubstituted alkyl-aryl; or C(O)—R with R being an optionally at least mono-substituted aryl,
R⁵ represents a hydrogen atom; or a linear or branched, saturated or unsaturated C₁₋₁₀ aliphatic radical which may be substituted with 1, 2 or 3 substituent(s) independently selected from the group consisting of F, Cl, Br, —OH, —NH₂, —SH, —O—CH₃, and —O—C₂H₅;
or
R² and R⁵ together with the bridging nitrogen form a saturated, unsaturated or aromatic 5- to 7-membered heterocyclic ring which may be substituted with 1, 2 or 3 substituent(s) independently selected from the group consisting of C₁₋₅-alkyl, —O—C₁₋₅-alkyl, —S—C₁₋₅-alkyl, oxo (═O), thioxo (═S), F, Cl, Br, I, —CN, —CF₃, —OCF₃, —OH, —SH, —NH₂, —CF₂H, —CFH₂, cyclopropyl, cyclobutyl, cyclopentyl, cyclohexyl, phenyl, phenoxy and benzyl and which may contain 1, 2 or 3 additional heteroatom(s) independently selected from the group consisting of nitrogen, oxygen and sulfur as a ring member(s) and which may be condensed with an unsaturated or saturated mono- or bicyclic ring system, which may be substituted with 1, 2 or 3 substituent(s) independently selected from the group consisting of C₁₋₅-alkyl, —O—C₁₋₅-alkyl, F, Cl, Br, I, —CN, —CF₃, —OCF₃, —SCF₃, —OH, —SH, —NH₂, —CF₂H, —CFH₂, and whereby the rings of the ring system are 5-, 6- or 7-membered and may contain 1, 2 or 3 heteroatom(s) independently selected from the group consisting of nitrogen, oxygen and sulfur;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

31. A combination of active substances according to claim 31, wherein the compound/s binding to the 5HT6-receptor is/are selected from the following group of sulfonamide compounds according to formula (Ie) consisting of:
[1] N-(1-(2-Dimethylamino)ethyl)-1H-indazol-6-yl)napthalene-2-sulphonamide;
[2] 5-Chloro-N-(1-(2-(dimethylamino)ethyl 1H-indazol-6-yl)-3-methylbenzo[b]thiophene-2-sulfonamide;
[3] Naphthalene-2-sulfonic acid [3-(1-methyl-piperidin-4-yl)-1H-indazol-5-yl]-amide,
[4] 5-Chloro-3-methyl-benzo[b]thiophene-2-sulfonic acid [3-(1-methyl-piperidin-4-yl)-1H-indazol-5-yl]-amide,
[5] Naphthalene-1-sulfonic acid [3-(1-methyl-piperidin-4-yl)-1H-indazol-5-yl]-amide,
[6] 4-Phenylbenzene-4-sulfonic acid [3-(1-methyl-piperidin-4-yl)-1H-indazol-5-yl]-amide,
[7] N-[3-(1-Methyl-piperidin-4-yl)-1H-indazol-5-yl]-4-phenoxy-benzenesulfonamide
[8] N-[3-(1-Methyl-piperidin-4-yl)-1H-indazol-5-yl]-benzenesulfonamide;
[9] N-[1-(2-Dimethylamino)ethyl)-2,3-dihydro-1H-indol-6-yl]-6-chloro-imidazo[2,1-b]thiazol-5-sulfonamide;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

32. A combination of active substances according to any of claims 1 to 8, wherein at least one compound (A) or at least one compound (B) or at least one compound (A) and one compound (B) binding to the 5HT6-receptor is/are selected from compounds according to formula (II) 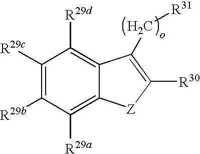
wherein
o is 0, 1, 2, 3 or 4
R³¹ represents a saturated or unsaturated cycloaliphatic radical, optionally at least monosubstituted, optionally at least with one heteroatom selected from N, o and S as a member of the ring that may be condensed with a mono or polycyclic annular system optionally at least monosubstituted; a —NR⁸R⁹ radical; a —CONR⁸R⁹ radical; —COOH; or —OH
where
R⁸ and R⁹ represent, independently of each other, a hydrogen atom; or a linear or branched, saturated or unsaturated C₁₋₅ aliphatic radical that may be substituted by 1, 2, 3 substituents selected independently from F, Cl, Br, —OH, —NH₂, —SH, —O—CH₃, —O—C₂H₅, —NO₂, —CN, —NH—CH₃ and —S—CH₃;
or R⁸ and R⁹ together with nitrogen form a saturated, unsaturated or aromatic heterocyclic ring with 3 to 9 members, which may be substituted by 1, 2 or 3 substituents selected independently from C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl and which may contain 1, 2 or 3 additional heteroatoms independently selected from N, O and S as members of the ring
R^{29a}, R^{29b}, R^{29c} and R^{29d} represent, independently of one another, a hydrogen atom; —NO₂; —NH₂; —SH; —OH; —CN; —C(═OYH; —C(═O)—R³¹; —OR³²; —SR³³; —SOR³⁴, —S(O)₂—R³⁴, —S(O)₂—N(R³⁵)R³⁶, —N(R³⁷S(O)₂—R³⁸; —NH—R³⁹; —NR⁴⁰R⁴¹; —N(R⁴²)—CO—R⁴³; F; Cl, Br; I; a linear or branched, saturated o unsaturated C₁-C₆ aliphatic radical, which may be substituted by 1, 2 or 3 substituents independently selected from F, Cl, Br, —OH, —NH₂, —SH, —O—CH₃, —O—C₂H₅, —NO₂, —CN, —NH—CH₃ and —S—CH₃; or an aryl or heteroaryl radical of 5 to 14 members, which may be substituted by 1, 2 or 3 substituents independently selected from —CF₃, C₁₋₅-alkyl, —O—C₁₋₅-alkyl, —S—C₁₋₅-alkyl, —C(O)—OH, —C(O)—O—C₁₋₅-alkyl, —O—C(O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(O)—C₁₋₅-alkyl, —N(C₁₋₅-alkyl)-C(O)—C₁₋₅-alkyl, —NO₂, —CHO, —CF₂H, —CFH₂, —C(O)NH₂, —C(O)—NH(C₁₋₅-alkyl), —C(O)—N(C₁₋₅-alkyl)₂, —S(O)₂—C₁₋₅-alkyl, —S(O)₂-phenyl, cyclopropyl, cyclobutyl, cyclopentyl, cyclohexyl, phenyl, phenoxy, benzyloxy and benzyl and which may be bonded by a linear or branched C₁-C₆ alkylene group, and where the heteroaryl radical contains 1, 2 or 3 heteroatoms independently selected from N, O and S as members of the ring;
with the condition that at least one of the substituents R^{29a}, R^{29b}, R^{29c} and R^{29d} represents a —NO₂, —SOR³⁴, —S(O)₂—R³⁴, —S(O)₂—N(R³⁵)R³⁶, —N(R³⁷)—S(O)₂—R³³, —N(R⁴²)—CO—R⁴³ radical;
Z represents: 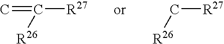
which respectively means (IIx) and (IIy) type compounds: 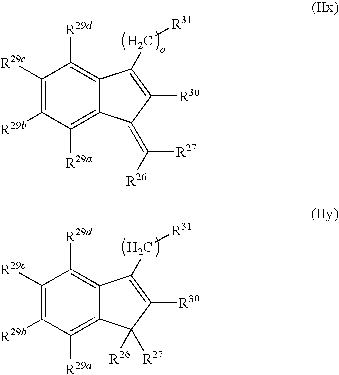
R²⁶ and R²⁷, identical or different, represent a hydrogen atom; NO₂; —NH₂; —SH; —OH; —CN; —C(═O)—R¹⁰; —OR¹¹; —SR¹²; F; Cl, Br; I; a linear or branched, saturated or unsaturated C₁-C₁₀ aliphatic radical, which may be substituted with 1, 2 or 3 substituents independently selected among F, Cl, Br, —OH, —SH, —O—CH₃, —O—C₂H₅, —NO₂, —CN and —S—CH₃; or an aryl or heteroaryl radical of 5 to 14 members, which may be substituted by 1, 2 or 3 substituents independently selected from —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy, benzyloxy and benzyl and which may be bonded by a linear or branched C₁-C₆ alkylene, C₂-C₆ alkenylene or C₁-C₆ ylidene group, and where the heteroaryl radical contains 1, 2 or 3 heteroatoms independently selected from N, O and S as members of the ring;
R³⁰ represents a hydrogen atom, a linear or branched C₁-C₆ aliphatic radical which may be substituted with 1, 2 or 3 substituents independently selected from F, Cl, Br, —OH, —SH, —O—CH₃, —O—C₂H₅, —NO₂, —CN and —S—CH₃;
R³¹ to R⁴³ represent, independently of each other, a hydrogen atom; a linear or branched, saturated or unsaturated C₁-C₅ aliphatic radical, which may be substituted by 1, 2 or 3 substituents independently selected from F, Cl, Br, —OH, —NH₂, —SH, —O—CH₃, —O—C₂H₅, —NO₂, —CN, —NH—CH₃ and —S—CH₃; a saturated or unsaturated cycloaliphatic radical with 3 to 8 members, which may be substituted by 1, 2 or 3 substituents independently selected from C₁₋₅-alkyl, —O—C₁₋₅-alkyl, —S—C₁₋₅-alkyl, oxo (═O), thioxo (═S), —C(═O)—OH, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy benzyloxy and benzyl and which optionally may include 1, 2 or 3 heteroatoms independently selected from N, O and S as members of the ring and which may be bonded through a linear or branched C₁-C₆ alkylene group; or an aryl or heteroaryl radical with 5 to 14 members that may be substituted by 1, 2 or 3 substituents independently selected from —CF₃, C₁₋₅-alkyl, —O—C₁₋₅-alkyl, —S—C₁₋₅-alkyl, —C(═O)—OH, —C(═O)—O—C₁₋₅-alkyl, —O—C(═O)—C₁₋₅-alkyl, F, Cl, Br, I, —CN, —OCF₃, —SCF₃, —OH, —SH, —NH₂, —NH(C₁₋₅-alkyl), —N(C₁₋₅-alkyl)₂, —NH—C(═O)—C₁₋₅-alkyl, —N(C₁₋₅-alkyl)-C(═O)—C₁₋₅-alkyl, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, cyclopropyl, cyclobutyl, cyclopentyl, cyclohexyl, phenyl, phenoxy, benzyloxy and benzyl and which may be bonded through a linear or branched C₁-C₆ alkylene, C₂-C₆ alkenylene or C₂-C₆ alkynylene group, and where the heteroaryl radical contains 1, 2 or 3 heteroatoms independently selected from N, O and S as members of the ring;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

33. A combination of active substances according to claim 32, wherein the compound/s binding to the 5HT6-receptor is/are selected from
[1] (2-methyl-6-nitro-3H-inden-1-yl)acetic acid
[2] [2-methyl-6-(naphthalene-2-sulphonylamine)-3H-inden-1-yl]acetic acid
[3] [3(Z)-benzylidene-2-methyl-6-(naphthalene-2-sulphonylamine)-3H-inden-1-yl]acetic acid
[4] [2-methyl-4-(naphthalene-2-sulphonylamine)-3H-inden-1-yl]acetic acid
[5] [6-(naphthalene-2-sulphonylamine)-3H-inden-1-yl]acetic acid
[6] [6-(5-chloro-3-methylbenzo[b]thiophene-2-sulphonylamine]-2-methyl-3H-inden-1-yl]acetic acid
[7] [2-methyl-6-(naphthalen-1-ylsulfamoyl)-3H-inden-1-yl]acetic acid
[8] N,N-Dimethyl-2-(2-methyl-6-nitro-3H-inden-1-yl)acetamide
[9] 2-2-Methyl-6-nitro-3H-inden-1-yl)-1-pyrrolidin-1-ylethanone
[10] 2-[3(Z)-Benzylidene-2-methyl-6-(naphthalene-2-sulphonylamine)-3H-inden-1-yl]-N,N-dimethylacetamide
[11] N,N-Dimethyl-2-[2-methyl-6-(naphthalene-2-sulphonylamine)-3H-inden-1-yl]acetamide
[12] N-[2-Methyl-3-(2-oxo-2-pyrrolidin-1-ylethyl)-1H-inden-5-yl]naphthalene-2-sulfonamide
[13] N-[2-Methyl-1-(2-oxo-2-pyrrolidin-1-ylethyl)-3H-inden-4-yl]naphthalene-2-sulfonamide
[14] N-[3-(2-Oxo-2-pyrrolidin-1-ylethyl) 1H-inden-5-yl]naphthalene-2-sulfonamide
[15] N-[2-Methyl-3-(2-oxo-2-pyrrolidin-1-ylethyl)-1H-inden-5-yl]-5-chloro-3-methyl benzo[b]thiophene-2-sulfonamide
[16] N,N-Dimethyl-2-[2-methyl-6-(naphthalen-1-ylsulfamoyl)-3H-inden-1-yl]acetamide
[17] Dimethyl-[2-(2-methyl-6-nitro-3H-inden-1-yl)ethyl]amine
[18] 3-(2-Dimethylaminoethyl)-2-methyl-1H-inden-5-ylamine
[19] N-[3-(2-Dimethylaminoethyl)-2-methyl-1H-inden-5-yl]-6-chloroimidazo[2,1-b]thiazole-5-sulfonamide
[20] N-[3-(2-Dimethylaminoethyl)-2-methyl-1H-inden-5-yl]-5-chloro-3-methylbenzo[b]thiophene-2-sulfonamide
[21] N-{4-[3-(2-Dimethylaminoethyl)-2-methyl-1H-inden-5-ylsulfamoyl]phenyl}acetamide
[22] N-[3-(2-Dimethylaminoethyl)-2-methyl-1H-inden-5-yl]benzo[1,2,5]thiadiazole-4-sulfonamide
[23] N-Ethyl-N-[3-(2-dimethylaminoethyl)-2-methyl-1H-inden-5-yl]-5-chloro-3-methylbenzo[b]thiophene-2-sulfonamide
[24] 4-Amino-N-[3-(2-dimethylaminoethyl)-2-methyl-1H-inden-5-yl]benzene sulfonamide
[25] N-[3-(2-Pyrrolidin-1-ylethyl)-2-methyl-1H-inden-5-yl]-2-(4-benzyloxyphenyl)acetamide
[26] 2-Methyl-3-(2-pyrrolidin-1-ylethyl)-1H-inden-5-ylamine
[27] (2-{6-[(5-chloro-3-methylbenzo[b}thiophene-2-sulfonyl)ethylamino]-2-methyl-3H-inden-1-yl}ethyl)ethyldimethylammonium iodide
[28] 1-[2-(2-Methyl-6-nitro-3H-inden-1-yl)ethyl]pyrrolidine
[29] N-[3-(2-Pyrrolidin-1-ylethyl)-2-methyl-1H-inden-5-yl]-6-chloroimidazo[2,1-b]thiazole-5-sulfonamide
[30] N-{4-[3-(2-Pyrrolidin-1-ylethyl)-2-methyl-1H-inden-5-ylsulfamoyl]phenyl}acetamide
[31] N-[3-(2-Pyrrolidin-1-ylethyl)-2-methyl-1H-inden-5-yl]-benzo[1,2,5]thiadiazole-4-sulfonamide
[32] 4-Amino-N-[3-(2-pyrrolidin-1-ylethyl)-2-methyl-1H-inden-5-yl]benzenosulfonamide
[33] N-[1 (Z)-Benzylidene-3-(2-dimethylaminoethyl)-2-methyl-1H-inden-5-yl]naphthalene-2-sulfonamide
[34] N-[3-(2-Dimethylaminoethyl)-2-methyl-1H-inden-5-yl]naphthalene-2-sulfonamide
[35] N-[2-Methyl-3-(2-pyrrolidin-1-ylethyl)-1H-inden-5-yl]naphthalene-2-sulfonamide
[36] N-[2-Methyl-1-(2-pyrrolidin-1-ylethyl)-3H-inden-4-yl]naphthalene-2-sulfonamide
[37] N-[3-(2-Pyrrolidin-1-ylethyl)-1H-inden-5-yl]naphthalene-2-sulfonamide
[38] N-[2-Methyl-3-(2-pyrrolidin-1-ylethyl)-1H-inden-5-yl]-5-chloro-3-methylbenzo[b]thiophene-2-sulfonamide
[39] N-(Naphthalen-1-yl)-3-(2-dimethiylaminoethyl)-2-methyl-1H-indeno-5-sulfonamide
[40] N-[3-(2-Hydroxyethyl)-2-methyl-1H-inden-5-yl]naphthalene-2-sulfonamide
[41] 6-Chloro-N-{3-[2-(dimethylamino)ethyl]-1,1-dimethyl-1H-inden-5-yl}imidazo[2,1-b] [1,3]thiazole-5-sulfonamide
[42] 5-Chloro-N-{3-[2-(dimethylamino)ethyl]-1,1-dimethyl-1H-inden-5-yl}-3-methylbenzo[b]thiophene-2-sulfonamide
[43] N-{3-[2-(Dimethylamino)ethyl]-2-methyl-1H-inden-5-yl}naphthalene-1-sulfonamide
[44] N-{3-[2-(Dimethylamino)ethyl]-2-methyl-1H-inden-5-yl}-1-benzothiophen e-3-sulfonamide
[45] 6-Chloro-N-[2-methyl-3-(1-methylpyrrolidin-3-yl)-1H-inden-5-yl]imidazo[2,1-b] [1,3]thiazole-5-sulfonamide
[46] 6-Chloro-N-[2-methyl-3-(1-methylpiperidin-3-yl)-1H-inden-5-yl]imidazo[2,1-b] [1,3]thiazole-5-sulfonamide
[48] 6-Chloro-N-{3-[2-(dimethylamino)ethyl]-1H-inden-5-yl}imidazo[2,1-b] [1,3]thiazole-5-sulfonamide
[49] 6-Chloro-N-[3-(2-piperidin-1-ylethyl)-1H-inden-5-yl]imidazo[2,1-b] [1,3]thiazole-5-sulfonamide
[50] 6-Chloro-N-[3-(1-methylpyrrolidin-3-yl)-1H-inden-5-yl]imidazo[2,1-b] [1,3] thiazole-5-sulfonamide;
optionally in form of one of its stereoisomers, preferably enantiomers or diastereomers, its racemate or in form of a mixture of at least two of its stereoisomers, preferably enantiomers or diastereomers, in any mixing ratio, or a salt, preferably a physiologically acceptable salt thereof, or a corresponding solvate, respectively.

34. A combination of active substances according to any of claims 1 to 8, wherein at least one compound (A) or at least one compound (B) or at least one compound (A) and one compound (B) binding to the 5HT6-receptor is/are selected from compounds according to formula (III) 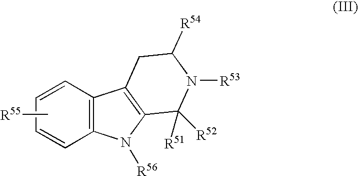
wherein
R⁵¹ and R⁵², identical or different, represent hydrogen, C₁-C₆ alkyl, C₂-C₆ alkenyl, C₂-C₆ alkinyl, aryl, heteroaryl, C₃-C₆ cycloalkyl or C₃-C₆ heterocycloalkyl, optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH-R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, independent from one another, each represent a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene, alkenylene or alkinylene group,
or R⁵¹ and R⁵² together form a spiro substituent of 3-6 carbons;
R⁵³ represents hydrogen, C₁-C₆ alkyl, C₂₋₆ alkenyl, C₂₋₆ alkinyl, C₃-C₆ cycloalkyl, C₃-C₆ heterocycloalkyl, aryl or heteroaryl; optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R)—S(═O)₂—R^{g}; —NH-R^{f}; —NRR^{f}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)R^{f}; —NR^{f}— C(═O)—R^{g}; —NH—C(═O)—O—R^{f}; —NR^{f}—C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
R⁵⁴ represents hydrogen, CO—NR^{a}R^{b}, CO—OR^{a}, wherein
R^{a} and R^{b}, identical or different, represent hydrogen, C₁-C₆ alkyl, aryl, heteroaryl, C₃-C₆ cycloalkyl, or C₃-C₆ heterocycloalkyl, optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH—R^{f}; —NR^{f}R^{g}; —C(═O)—NHR^{f}, —C(═O)NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; an halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
R⁵⁵ represents NRCSO₂R^{d}, wherein
R^{c} represents hydrogen or C₁₋₄ alkyl optionally substituted with one or more substituents independently selected from C₁-C₆ alkyl, aryl, cyano, C₁-C₆ alkoxy and trifluoromethyl;
R^{d} represents aryl or heteroaryl optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R)—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; a halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
R⁵⁶ represents hydrogen, C₁₋₄ alkyl, aryl, heteroaryl or SO₂R^{e}, wherein
R^{e} represents aryl, heteroaryl, C₃-C₆ cycloalkyl, C₃-C₆ heterocycloalkyl; optionally substituted with one or more substituents independently selected from —NO₂; —NH₂; —SH; —OH; —CN; —C(═O)—OH; —S(═O)₂—OH; —C(═O)—NH₂; —S(═O)₂—NH₂; —S(═O)₂—R^{f}; —OR^{f}; —SR^{f}; —C(═O)—OR^{f}; —N(R^{f})—S(═O)₂—R^{g}; —NH—R^{f}; —NR^{f}R^{g}; —C(═O)—NHR^{f}, —C(═O)—NR^{f}R^{g}; —S(═O)₂—NHR^{f}, —S(═O)₂—NR^{f}R^{g}; —O—C(═O)—R^{f}; —NH—C(═O)—R^{f}; —NR^{f}—C(═O)—R^{g}; —NH—C(═O)—O—R^{f}; —NR^{f}—C(═O)—O—R^{g}; —S(═O)₂—O—R^{f}; an halogen atom; a linear or branched, saturated or unsaturated, optionally at least mono-substituted aliphatic radical; a saturated or unsaturated, optionally at least mono-substituted, optionally at least one heteroatom as a ring member containing cycloaliphatic radical, which may be bonded via a linear or branched alkylene group; or an optionally at least mono-substituted aryl or heteroaryl radical, which may be bonded via a linear or branched alkylene group;
wherein R^{f} and R^{g}, have the meaning defined above
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

35. A combination of active substances according to claim 34, wherein the compound/s binding to the 5HT6-receptor is/are selected from
[1] 6-chloro-imidazo[2,1-b]thiazole-5-sulfonic acid (2-methyl-2,3,4,9-tetrahydro-1H-β-carbolin-6-yl)-amide;
[2] Benzo[b]thiophene-3-sulfonic acid (2-methyl-2,3,4,9-tetrahydro-1H-β-carbolin-6-yl)-amide;
[3] Naphthalene-1-sulfonic acid (2-methyl-2,3,4,9-tetrahydro-1H-β-carbolin-6-yl)-amide;
[4] 5-Chloro-naphthalene-2-sulfonic acid (2-methyl-2,3,4,9-tetrahydro-1H-β-carbolin-6-yl)-amide;
[5] 5-Chloro-3-methyl-benzo[b]thiophene-2-sulfonic acid (2-methyl-2,3,4,9-tetrahydro-1H-β-carbolin-6-yl)-amide;
[6] Benzo[1,2,5]thiadiazole-4-sulfonic acid (2-methyl-2,3,4,9-tetrahydro-1H-β-carbolin-6-yl)-amide;
[7] N-[4-2-Methyl-2,3,4,9-tetrahydro-1H-β-carbolin-6-ylsulfamoyl)-phenyl]-acetamide;
[8] 4-Amino-N-(2-methyl-2,3,4,9-tetrahydro-1H-β-carbolin-6-yl)-benzenesulfonamide;
[9] N-[4-Methyl-5-(2-methyl-2,3,4,9-tetrahydro-1H-β-carbolin-6-ylsulfamoyl)-thiazol-2-yl]-acetamide;
[10] 5-Dimethylamino-naphthalene-1-sulfonic acid (2-methyl-2,3,4,9-tetrahydro-1H-β-carbolin-6-yl)-amide;
[11] Benzofuran-2-sulfonic acid (2-methyl-2,3,4,9-tetrahydro-1H-β-carbolin-6-yl)-amide;
[12] Naphthalene-2-sulfonic acid (2-methyl-2,3,4,9-tetrahydro-1H-β-carbolin-6-yl) amide;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

36. Medicament comprising a combination of active substances according to any of claim 1 to 35 and optionally at least one pharmaceutical adjuvants.

37. Medicament according to claim 36 for the treatment of Peripheral Nervous System Disorders, or Central Nervous System Disorders, especially Central Nervous System Disorders.

38. Medicament according to any of claims 36 or 37 for the treatment of cognitive disorders, memory disorders, senile dementia processes, such as Alzheimer's, Parkinson's and/or Huntington's Disease, attention deficit disorder, such as infantile hyperkinesia (ADHD, attention deficit/hyperactivity disorder), epilepsy, anxiety, panic, depression, psychosis, pain, schizophrenia; or for the improvement/enhancement of cognition.

39. Medicament according to any of claims 36 to 38 for the treatment of cognitive disorders, degenerative disorders, memory disorders, ADHD (attention deficit/hyperactivity disorder), Alzheimer's disease, senile dementia process, learning disabilities caused by degenerative disorders, learning disabilities caused by non-degenerative disorders, memory or cognitive dysfunction such as mild cognitive impairment, age-related cognitive decline, cerebral senility, vascular dementia, AIDS-associated dementia, electric shock induced amnesia, memory impairment associated with depression or anxiety, cognitive defects in Parkinson's disease, Down's syndrome, stroke, traumatic brain injury, Huntington's disease, and attention deficit disorder, infantile hyperkinesia (ADHD, attention deficit/hyperactivity disorder); especially ADHD, or for the improvement/enhancement of cognition.

40. Use of a combination of active substances according to any of claims 1 to 35 for the manufacture of a medicament for the treatment of Peripheral Nervous System Disorders, or Central Nervous System Disorders, especially Central Nervous System Disorders.

41. Use according to claim 40, characterized in that the medicament is for the treatment of cognitive disorders, memory disorders, senile dementia processes, such as Alzheimer's, Parkinson's and/or Huntington's Disease, attention deficit disorder, such as infantile hyperkinesia (ADHD, attention deficit/hyperactivity disorder), epilepsy, anxiety, panic, depression, psychosis, pain, schizophrenia; or for the improvementenhancement of cognition.

42. Medicament according to any of claims 40 to 41, characterized in that the medicament is for the treatment of cognitive disorders, degenerative disorders, memory disorders, ADHD (attention deficit/hyperactivity disorder), Alzheimer's disease, senile dementia process, learning disabilities caused by degenerative disorders, learning disabilities caused by non-degenerative disorders, memory or cognitive dysfunction such as mild cognitive impairment, age-related cognitive decline, cerebral senility, vascular dementia, AIDS-associated dementia, electric shock induced amnesia, memory impairment associated with depression or anxiety, cognitive defects in Parkinson's disease, Down's syndrome, stroke, traumatic brain injury, Huntington's disease, and attention deficit disorder, infantile hyperkinesia (ADHD, attention deficit/hyperactivity disorder); especially ADHD, or for the improvement/enhancement of cognition.
